(19) United States
(12) Patent Application Publication
(10) Pub. No.: US 2017/0246486 A1
(43) Pub. Date: Aug. 31, 2017

(54) SYSTEMS AND METHODS FOR AIR FILTRATION MONITORING

(71) Applicant: Free Air, Inc., Boone, NC (US)

(72) Inventors: Joseph Cazier, Boone, NC (US); Shawn M. Bergman, Boone, NC (US); Charles Eric Hunter, Boone, NC (US); Iyam Lynch, Boone, NC (US); J. Sid Clements, Boone, NC (US); Bradley G. Johnson, Boone, NC (US)

(21) Appl. No.: 15/510,534

(22) PCT Filed: Sep. 14, 2015
(86) PCT No.: PCT/US2015/050033

§ 371 Date: Mar. 10, 2017

Related U.S. Application Data
(60) Provisional application No. 62/192,534, filed on Jul. 14, 2015.
(60) Provisional application No. 62/159,314, filed on May 10, 2015.
(60) Provisional application No. 62/049,862, filed on Sep. 12, 2014.

(51) Int. Cl. A62B 27/00; B01D 46/00; B01D 46/44; A62B 18/08; F24F 3/16; A61B 5/024; A61B 5/087; A61B 5/00; F24F 11/00; B01D 46/42; A62B 7/10

(52) U.S. Cl. CPC A62B 27/00; B01D 46/429; B01D 46/0002; B01D 46/0047; B01D 46/0026; B01D 46/44; A62B 18/088; A62B 7/10; A61B 5/02438; A61B 5/087; A61B 5/7282; F24F 11/0017; F24F 11/006; F24F 3/1603; B01D 2273/18

(57) ABSTRACT

Implementations described and claimed herein provide air filtration monitoring. In one implementation, air filtration data is received from one or more air filtration systems over a network. Each of the one or more air filtration systems is configured to provide purified air into an enclosed space by removing ultra-fine particles from air using at least one primary filter. The air filtration data is captured by one or more sensors. The air filtration data is correlated based on at least one monitoring parameter, and air filtration analytics are generated from the correlated data. In another implementation, health data is received from a controller in an air filtration system. The health data is captured using one or more sensors. Health monitoring analytics are generated from the health data, and feedback is generated from the health monitoring analytics.

### CROSS-REFERENCE TO RELATED APPLICATIONS

The present application claims benefit under 35 U.S.C. §119 to: U.S. Provisional Patent Application No. 62/049,862, entitled “Personal Respirators and Air Filtration Systems with Data Capture and Data Analytics Thereto” and filed on Sep. 12, 2014; U.S. Provisional Patent Application No. 62/159,314, entitled “Small, Lightweight, Low Power, Personal Respirator with Low Face Velocity to Remove Ultrafine Particles” and filed on May 10, 2015; and U.S. Provisional Patent Application No. 62/192,534, entitled “Small, Lightweight, Low Power, Personal Respirator with Low Face Velocity to Remove Ultrafine Particles” and filed on Jul. 14, 2015, each of which is incorporated by reference in its entirety herein.

### TECHNICAL FIELD

Aspects of the present disclosure relate to air filtration monitoring and more particularly to monitoring health and environmental air quality, among other parameters, using one or more air filtration systems.

### BACKGROUND

Air pollution is a serious and complex global problem. Long term exposure can lead to a variety of negative health consequences (e.g., loss of lung capacity, asthma, bronchitis, emphysema, and possibly some forms of cancer). Millions of deaths occur each year as a result of air pollution exposure. While air pollution is generally defined as airborne particles that are less than 10 microns in diameter (“PM10” class), the most dangerous class of airborne particulate pollution is the PM2.5 class, which includes pollutant particles that are less than 2.5 microns in diameter. Ultra-fine particles (“UFPs”) that are less than 0.1 microns (100 nm) pose serious health risks with the potential of enhanced toxicity and contribution to health effects beyond the respiratory system. Airborne diseases, such as bacterial or viral diseases, also present worldwide health issues. Such issues are especially concerning where a highly communicable, serious or life threatening disease emerges and spreads in a population, particularly if the disease is resistant to treatment or difficult to treat with existing therapies.

Conventional systems may measure a current pollution level within a geographic area, such as a city. However, such measurements are often not indicative of the quality of air that users within that area are actually breathing. For example, many users rely on air filtration systems to purify the air prior to inhalation. Conventional systems are generally deployed within the geographical area to monitor a quality of the ambient air and thus lack the ability to monitor a quality of the purified air that users are breathing.

Individuals with a decreased lung capacity or who suffer from a respiratory condition may be particularly susceptible to air pollution and/or airborne disease exposure. Diagnosis and monitoring respiratory conditions can be particularly challenging in areas plagued with low air quality. Moreover, individuals with decreased lung capacity may be sensitive to high air flow, emphasizing an importance of monitoring operational parameters of air filtration devices.

It is with these observations in mind, among others, that various aspects of the present disclosure were conceived and developed.

### SUMMARY

Implementations described and claimed herein address the foregoing problems by providing systems and methods for air filtration monitoring. In one implementation, air filtration data is received from one or more air filtration systems over a network. Each of the one or more air filtration systems is configured to provide purified air into an enclosed space by removing ultra-fine particles from air using at least one primary filter. The air filtration data is captured by one or more sensors. The air filtration data is correlated based on at least one monitoring parameter, and air filtration analytics are generated from the correlated data.

In another implementation, health data is received from a controller in an air filtration system configured to provide purified air into an enclosed space by removing ultra-fine particles from air using at least one primary filter. The health data is captured using one or more sensors. Health monitoring analytics are generated from the health data, and feedback is generated from the health monitoring analytics.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 illustrates an air monitoring system, including a monitor which may run on a computer server, computing device, or other network device, for air monitoring using one or more air filtration systems.

FIG. 2 illustrates an example air filtration system including a powered air purifying respirator fitted to a user during operation.

FIG. 3 illustrates another example air filtration system including a room air cleaner.

FIGS. 4A and 4B depict a side perspective view and a back view, respectively, of an example powered air purifying respirator.

FIG. 5 illustrates an interior view of the powered air purifying respirator of FIGS. 4A-B.

FIGS. 6A and 6B are front and side views, respectively, of air flow through the filter module of FIGS. 4A-B.

FIG. 7 illustrates air flow paths through the respirator of FIGS. 4A-B into a mask.

FIGS. 8A and 8B show a top perspective view and a bottom perspective view, respectively, of an example room air cleaner.

FIG. 9 is a cross-sectional view illustrating air flow through the room air cleaner of FIGS. 8A-B.

FIG. 10 is an example personal respiratory health user interface.

FIG. 11 is an example respiratory health user interface for monitoring breathing patterns.

FIG. 12 is an example air filtration analytics user interface.

FIG. 13 is a block diagram of an example air filtration system.

FIG. 14 illustrates example operations for air filtration monitoring.

FIG. 15 is a functional block diagram of an electronic device including operational units arranged to perform air filtration monitoring operations.

FIG. 16 illustrates example operations for health monitoring.

FIG. 17 is a functional block diagram of an electronic device including operational units arranged to perform health monitoring operations.

FIG. 18 is an example computing system that may implement various systems and methods of the presently disclosed technology.

### DETAILED DESCRIPTION

Aspects of the present disclosure generally relate to system and methods for air filtration monitoring using one or more air filtration systems configured to remove ultra-fine particles (UFPs) to provide purified air into an enclosed space. In one aspect, the air filtration systems each comprise one or more sensors configured to capture air filtration data and/or health data. Using this data, analytics may be generated pertaining to operational parameters of the air filtration system, ambient air quality, purified air quality, user health, and/or the like. The analytics may be output, for example, for display on a user device and/or feedback may be generated from the analytics.

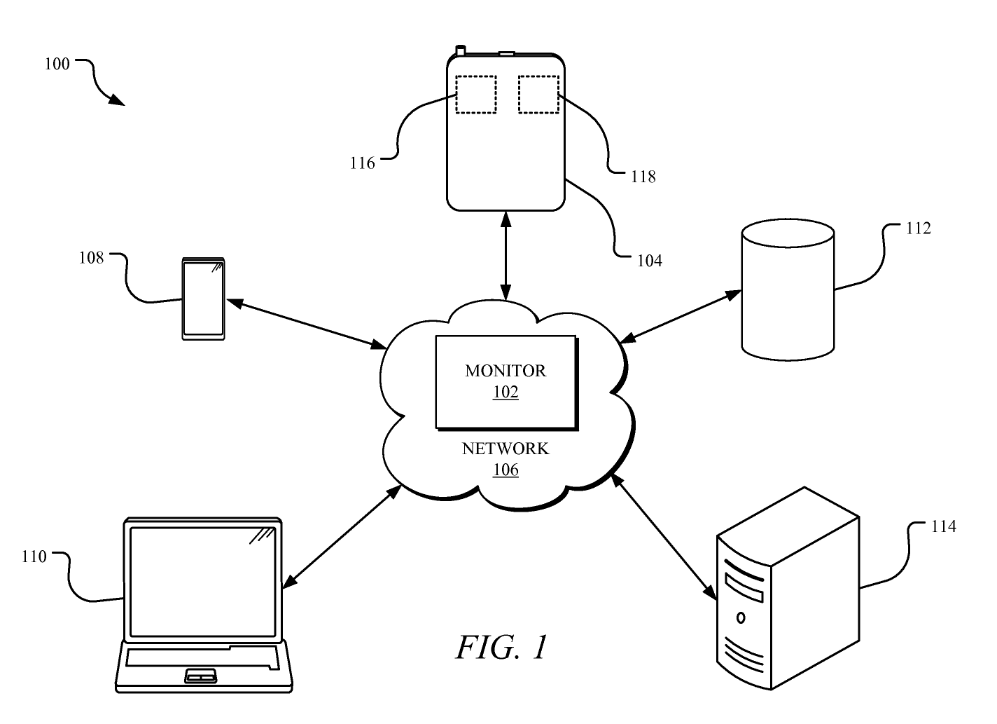

FIG. 1 is an example air monitoring system 100, including a monitor 102 running on a computer server, computing device, or other network device, for air filtration monitoring. In one implementation, a user accesses and interacts with the monitor 102 and/or one or more air filtration systems 104 via a network 106 (e.g., the Internet). In another implementation, a user device (e.g., a consumer device 108, an administrator device 110, etc.) locally runs the monitor 102, and the air filtration system(s) 104 connect to the user device using a wired or wireless connection. The user may be, without limitation, a consumer, an administrator, and/or the like. The consumer may be one or more end users of the air filtration systems 104, and the administrator may be one or more parties that sell, operate, manage, and/or otherwise monitor the air filtration systems 104, including a physician, health clinic, health laboratory, and/or the like.

The network 106 is used by one or more computing or data storage devices (e.g., one or more databases 112) for implementing the air monitoring system 100. The user may access and interact with the monitor 102 using a user device, such as the consumer device 108 or the administrator device 110, communicatively connected to the network 106. The user device is generally any form of computing device capable of interacting with the network 106, such as a desktop computer, workstation, terminal, portable computer, mobile device, smartphone, tablet, multimedia console, and/or the like.

A server 114 may host the air monitoring system 100. The server 114 may also host a website or an application, such as the monitor 102 that the user visits to access the system 100. The server 114 may be one single server, a plurality of servers with each such server being a physical server or a virtual machine, or a collection of both physical servers and virtual machines. In another implementation, a cloud hosts one or more components of the system 100. The one or more air filtration systems 104, the user devices employed by the consumer 108 and the administrator 110, the server 114, and other resources, such as the one or more databases 112, connected to the network 106 may access one or more other servers for access to one or more websites, applications, web services interfaces, etc. that are used for air filtration monitoring. The server 114 may also host a search engine that the air monitoring system 100 uses for accessing and modifying information used for air filtration monitoring.

The air filtration systems 104 communicate with the monitor 102 executed by the consumer device 108 and/or the administrator device 110 via a wireless connection, such as Bluetooth, over the network 106, or via a wired connection, such as a USB connection. The air filtration systems 104 may communicate in similar manners with other computing devices, such as a smart watch, smartphone, tablet, computer, music player, Bluetooth enabled devices, and the like.

In one implementation, the air filtration systems 104 include one or more sensors 116 for capturing health data and/or air filtration data. The sensors 116 may include, without limitation, one or more pressure sensors, humidity sensors, temperature sensors, particle sensors, heart rate sensors, carbon dioxide sensors, oxide sensors, ozone sensors, nitric oxide sensors, microphones, imaging sensors, and/or the like. Such data may be stored in storage media of the air filtration systems 104 and/or communicated to the monitor 102 using a controller 118. By way of example, the data captured by the sensors 116 may be retrieved and stored on the consumer device 108 or the administrator device 110 and/or uploaded to a secure cloud over the network 106 to the databases 112.

Once the data is obtained by the monitor 102, it can be utilized in many ways by the user and other approved parties. For example, a healthcare professional may access the monitor 102 with the administrator device 110 to monitor user compliance with a prescribed air filtration regimen. In some implementations, the monitor 102 obtains health data, including usage data, such as the day, time, and duration that the air filtration system 104 has been operating. Other health data may include data pertaining to: use of the air filtration system 104, data indicative of a condition or health of the consumer, diagnoses, treatment effectiveness, user symptoms, and/or the like. In one implementation, the administrator accesses the health data for one or more consumers by logging into the monitor 102 with the administrator device 110. The consumer may provide access to the administrator using settings of the monitor 102. The heath data is valuable to the administrator because in forming a medical recommendation to the consumer as well as to evaluate the role the air filtration system 104 plays in improving the consumer's health.

In one implementation, the monitor 102 obtains a heart rate measurement, air flow pressure data, and other health data from the sensors 116. The monitor 102 correlates the air flow pressure data with breathing patterns to generate health monitoring analytics, including predictions related to the users current and/or future health condition.

The health monitoring analytics generated by the monitor 102 may be used to monitor or indirectly infer various health conditions of the consumer. The basic concept of correlating health data from the sensors 116 to generate the health data, includes the monitor 102 analyzing consumer baseline physical and health conditions, such as a breathing curve (inhalation and exhalation pressure response) over time. In one implementation, the monitor 102 develops criteria for normal conditions, such breathing patterns, over a set period of time. To increase the statistical power of the measurement technique, the monitor 102 may utilize numerous amounts of data over relatively long periods of time for multiple consumers in controlled environmental conditions at specified activity levels.

In one implementation, the data captured by the sensors 116 pertaining to air filtration may be coupled with a heart rate reading over time for monitoring the health and/or athletic performance. Health data collected from the sensors 116 regarding pressure may be used to directly monitor or indirectly infer breathing patterns of the consumer. In one implementation, the monitor 102 uses the health data, including pressure data, to measure forced exhalation volume (FEV1). Normal breathing is relative to the consumer's baseline activity level and as a result there may be multiple “normal breathing” settings based on an activity of the consumer. Nonetheless, once the baseline “normal breathing” pattern is established, the monitor 102 may generate health monitoring analytics based on abnormalities in breathing pattern distinguished from the baseline to differentiate between healthy and unhealthy conditions of the consumer.

The health monitoring analytics may further relate to calibration and air flow of the air filtration system 104, diagnosis of conditions (e.g., asthma or COPD), monitoring of conditions, testing (e.g., spirometry testing), symptoms monitoring (e.g., respiratory symptoms monitoring), and/or the like.

The monitor 102 may generate real-time feedback, including alerts to the administrator device 110, the consumer device 106, and/or the air filtration system 104 regarding a health condition of the consumer. The monitor 102 may generate feedback in the form of suggested or automatic changes to operational parameters of the air filtration system 104. For example, increased air pressure inside of a breathing mask delivered to an individual suffering with a lung abnormality such as chronic obstructive pulmonary disease (COPD) can greatly improve breathing. The excess pressure inside of the mask helps open up the individual's lungs which in effect reduces the work of breathing for those with weak performing lungs. Thus, the monitor 102 may increase the pressure by approximately 10 cm of water (3.93 inches of water) or another amount by communicating with the controller 118 of the air filtration system 104 and monitoring the effect with the sensors 116. The monitor 102 may send commands to the controller 118, for example, the increase pressure inside of the mask by altering an exhalation valve diameter and durometer to a smaller hole and stiffer valve. These changes allow the mask to retain a higher level of the air pressure generated by the device's fan. In certain implementations, the exhalation valve may be selected so as to result in a system that can exceed 3 cm H₂O under normal operation and 8 cm H₂O at maximum output. By way of illustration, effective valve diameter range for this application (9 mm-30 mm) and the stiffness of the exhalation valve can range from 40 A-70 A. In accordance with certain aspects of the disclosure, the achievable pressure range inside of the mask generally ranges from 1 cm H₂O-11 cm H₂O.

For instance, by way of non-limiting example, the pressure levels from a 17.5 mm diameter size allows the system to be used as a continuous flow CPAP machine with the added benefit of supplying highly purified air (higher than existing CPAP machines) to the user as they are undergoing treatment.

The monitor 102 may further generate feedback in the form of instructions to the controller 118 to deliver drug and active pharmaceutical ingredients to the consumer as the health monitoring analytics indicates. For example, if the health monitoring analytics generated by the monitor 102 indicate an asthma or COPD condition, the monitor 102 may instruct the controller 118 to operate the air filtration system 104 to administer an indicated amount of asthma medication (e.g., albuterol).

In one implementation, the monitor 102 obtains air filtration data quantifying the behavior of one or more operational aspects of the air filtration systems 104. The air filtration data may be captured from the sensors 116, correlated, and stored in the databases 112. Once gathered, the operational respirator/filtration data may be correlated according to at least one monitoring parameter (e.g., a parameter of the air filtration system 104 and/or of the consumer(s)) to generate air filtration analytics. The air filtration analytics may include, without limitation, respirator analytics for the air filtration systems 104 including operational data; use analytics, including consumer use patterns, product use research, use compliance, and extended use; health analytics, including environmental health and user health; device analytics, including connecting device operation and product performance; demographic analytics; media analytics, including social media, marketing, and social sharing; and/or the like. The monitor 102 may output the air filtration analytics to the consumer device 108, the administrator device 110, the air filtration systems 104, and/or the like the form of alerts, alarms, and/or other types of structured reporting.

In one implementation, the administrator is a manufacturer or manager of the air filtration systems 104, and the administrator accesses the air filtration analytics generated by the monitor 102 using the administrator device 110. Effective monitoring of the air filtration systems 104 enables the administrator to validate operational aspects of the air filtration systems 104, as well as the ability to review, analyze, and validate the health of consumers interacting with the air filtration systems 104.

The air filtration analytics may be used to analyze a microenvironment or microclimate of the consumer. Microenvironments or microclimates are generally localized atmospheric zones where the average pattern of variation in temperature, humidity, barometric pressure, particle count, and other ambient air factors differs from the surrounding area. Microclimates may be as small as a few square feet or as large as many square miles. Microclimates exist, for example, near bodies of water which may cool the local atmosphere; in and around urban areas where brick, concrete, and asphalt absorb the suds energy and building change wind patterns; around highway and road ways where vehicles produce various types of emissions and tires grind up and disperse particles; and in rural and agricultural areas where differences in vegetation contribute to different moisture, temperature, and particular concentration. In one implementation, the air filtration analytics are related to microclimates and microenvironments, both from a temporal and/or geospatial perspective.

In one implementation, the monitor 102 inspects, cleans, transforms, and/or models large amounts of the captured air filtration data, which may be structured or unstructured, to generate or otherwise discover useful information and/or correlations, suggest conclusions, and support business decision making. The monitor 102 may generate one or more discrete analytic values that may be used to quantify performance of the air filtration systems 104 from which the air filtration data was originally obtained. In one implementation, the monitor 102 processes air filtration data obtained from the sensors 116 to generate air filtration analytics that quantify some aspect of performance of the air filtration systems 104 and/or characteristics of the consumers. For example, the air filtration data may be processed and analyzed to: validate operational aspects of the respirator and/or air filtration systems; identify consumer use patterns corresponding to the respirator/air filtration systems; identify potential respirator/air filtration system performance improvements; perform respirator/air filtration system use-compliance and reporting; generate respirator/air filtration system environmental and health correlations, and/or the like.

It will be appreciated that the health monitoring analytics and/or the air filtration analytics may be generated according to at least one monitoring parameter, including a set of consumers (e.g., for one consumer or a group of consumers), one or more data types captured by the sensors 116 (e.g., pressure, temperature, particle detection, heart rate, etc.), one or more behavior patterns (e.g., behavior patterns of the consumers, operational patterns of the air filtration systems 104, etc.), a monitoring area (e.g., one or more of the enclosed spaces), an environmental monitoring area (e.g., one or more regions in which the air filtration systems 104 are deployed), and/or the like.

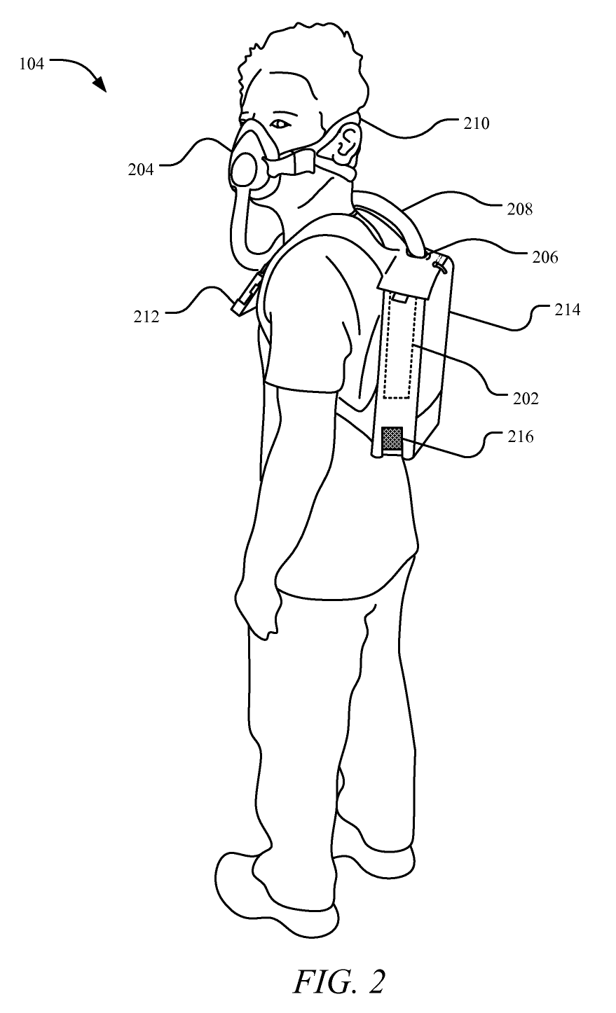

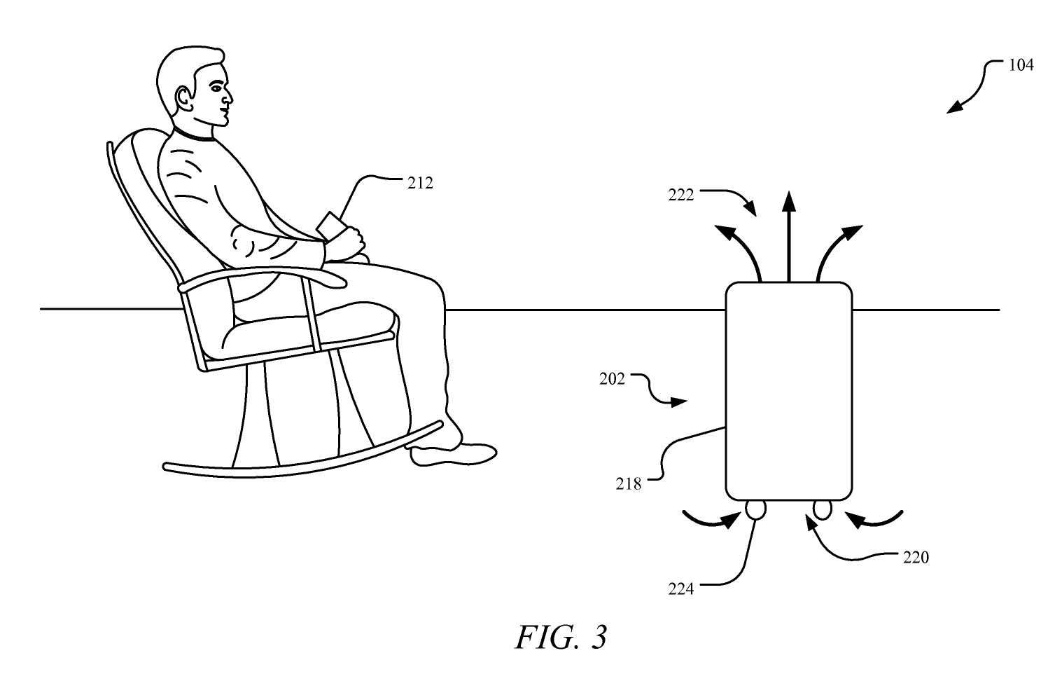

To begin a detailed description of examples of the air filtration systems 104, reference is made to FIGS. 2 and 3, which illustrate the air filtration system 104 including a powered air purifying respirator and a room air cleaner, respectively. It will be appreciated that the air filtration systems 104 shown in FIGS. 2-3 are exemplary only, and the air filtration systems 104 may include any devices for purifying air, including personal respirators, room air cleaners, heating, ventilating, and air conditioning (HVAC) systems, free standing systems, system integrated air filtration systems, and/or the like. The systems and methods of the air filtration systems 104 may be similar to those described in International Patent Application No. PCT/US2015/034260, entitled “Systems and Methods for Removing Ultra-Fine Particles from Air” and filed on Jun. 4, 2015, and/or International Patent Application No. PCT/US2015/039127, entitled “Room Air Cleaner Systems and Methods Related Thereto” and filed on Jul. 2, 2015. The entirety of each of these applications is incorporated by reference herein.

Turning first to FIG. 2, in one implementation, the air filtration system 104 includes an air purifier 202 in the form of a powered air purifying respirator configured for removing UFPs to provide filtered air to an enclosed space, which may be, without limitation, a mask 204 fitted to a user with one or more straps 210. The straps 210 may be provided in various orientations, including, without limitation, one or more head straps, a neck attachment along the jawline of a user, a helmet, and the like.

In one implementation, one or more hoses 208 connect the mask 204 to the air purifier 202 at an outlet 206. The hose 208 may be detachable from the mask 204 and/or the air purifier 202. In one implementation, the hose 208 tapers proximally from the air purifier 202 to the mask 204, permitting a lower pressure drop through the air filtration system 104.

The tapering of the hose 208 may also permit the hose 208 to extend through a strap of a carrying case 214, which may be, without limitation, a messenger bag, a briefcase, a backpack, a purse, and other bags or cases configured for facilitating carrying of the air purifier 202. A cover may wrap around the hose 208 prior to insertion into a strap of the carrying case 214. The cover may be formed, for example, from a spandex or similar material and include an attachment mechanism, such as paired hooks and loops.

The carrying case 214 may include various pockets, openings, access panels, and/or the like. For example, the carrying case 214 may include one or more vents 116 through which the air purifier 202 draws in outside air for filtration. In one implementation, the carrying case 214 includes a pocket or similar attachment mechanism to hold a user device 212, which may be the consumer device 108 or the administrator device 110. In another implementation, the user device 212 includes a case 120 with an attachment mechanism, such as a clip, latch, fastener, clasp, pin, hook, or the like for attaching the user device 212 to the carrying case 214 or the user.

The user device 212 is in communication with the air purifier 202 for controlling the operations of the air purifier 202. The user device 212 is generally any form of computing device, such as a mobile device, tablet, personal computer, multimedia console, set top box, or the like, capable of interacting with the air purifier 202. The user device 212 may communicate with the air purifier 202 via a wired (e.g., Universal Serial Bus (USB) cable 118) and/or wireless (e.g., Bluetooth or WiFi) connection. In addition to controlling the operation of the air purifier 202, the user device 212 may be used to monitor the performance of the air purifier 202, including filter and collection efficiency, power consumption, system pressure, air flow rates, and the like. The user device 212 further provides real time information on power level, fan speed, filter life, and pressure alarm.

In one implementation, the air purifier 202 achieves extremely high filter efficiencies below 10e-9 at low face velocities less than or equal to 5 cm/s. At such face velocities, the air purifier 202 has a filter efficiency of 99.99999% down to 0.01 microns. The air purifier 202 filters UFPs and (e.g., below 300 nm down to 10 nm and below), as well as pathogens of similar size. Conventional passive masks cannot achieve comparable filtration, due in part to the inhalation capacity of users. Smaller pore sizes in such passive masks would result in a large increase in the resistance a user would feel while attempting to draw air through the air purifier 202 during inhalation. Such passive masks, thus, cannot achieve comparable filter efficiencies for particle sizes below 300 nm. As a result, conventional passive masks fail to filter UFPs below 100 nm, which may diffuse through the alveoli in the lung into the bloodstream and deposit in the brain or other vital organs causing or exacerbating diseases such as dementia, Alzheimer's, and the like, as well as fail to prevent the intrusion of pathogens such as dangerous flu viruses, the common cold, and other pathogens that are less than 100 nm in size.

The air filtration system 104 incorporates positive air flow, which provides increased comfort during normal breathing and protects against contamination resulting from leakage paths around the mask 208 caused by instantaneous negative pressure gradients due to inhalation or gasping. For example, the air filtration system 104 may deliver positive pressure air at flow rates of between approximately 50 and 300 standard liters per minute (“SLM”).

Referring to FIG. 3, in one implementation, the air filtration system 104 includes the air purifier 202 in the form of a room air cleaner including a housing 218 having an air inlet 220, an air outlet 222, and a plurality of wheels 224 facilitating relocation of the air purifier 202. The air purifier 202 provides purified air to one or more users in a room or other enclosed space. The air purifier 202 may be used in a nursery to provide purified air to infant while permitting a user to monitor the infant's breathing, for example, via the user device 212.

In one implementation, the air inlet 220 draws ambient air from the room into the housing 218 for purification and recirculates purified air into the room via the air outlet 222. Stated differently, the air purifier 202 removes UFPs and airborne pathogens from the ambient air in the room and recirculates purified air into the room. In one implementation, the air purifier 202 separates air flow through the housing 218 into a filtration air flow and a recirculation air flow, thereby achieving high filtration and power efficiencies.

The air purifier 202 generates the filtration air flow at a lower rate than the recirculation air flow. The relatively lower air flow rate during filtration achieves a low face velocity at the primary filter, which provides a high filter efficiency. In certain implementations, the filtration air flow provided to the surface of the primary filter is provided a low face velocity, e.g., at a face velocity of less than 5 cm/s, less than 4 cm/s, less than 3 cm/s, less than 2 cm/s, less than 1 cm/s, etc. For example, during filtering, the filtration air flow has a particle efficiency down to 99.9999. Once the filtration air flow is reduced from approximately 400 cubic feet per minute (CFM) to 100 CFM across a primary filter in the air purifier 202, the particle face velocity drops to approximately 0.25 cm per second, where the filter efficiency is below 10⁻¹⁰. Because UFPs, which include particles below 100 nanometers in size, diffuse through the alveoli in the lungs and deposit in end organs, such as the brain and pancreases, the air purifier 202 filters rooms, such as the nursery 118, to levels below 10⁻¹⁰ for particles 10 nanometers and below. While the filtration air flow is generated at a lower rate to increase filtration efficiency, the recirculation air flow is maintained at a high rate to ensure that the filtered air is distributed throughout the room.

In addition to the separation of the filtration air flow from the recirculation air flow, the air purifier 202 achieves high efficiencies through the use of a high surface area membrane filter, the use of stacked axial filtration fans, and optionally remoting (separation and removal) of electronics in the air purifier 202 from the filtered air flow, as described herein. The high surface area membrane filter increases filtration efficiency, while the stacked axial filtration fans decrease power consumption by the air purifier 202 without sacrificing static pressure. Remoting the electronics from the filtered air flow eliminates or otherwise reduces a potential for volatile organic compound (VOCs) contamination from the electronics.

As described herein, the air filtration systems 104 shown in FIGS. 2 and 3 may have one or more sensors 116 and a controller 118 to monitor and/or control the operations of the air filtration systems 104, as well as monitor air quality.

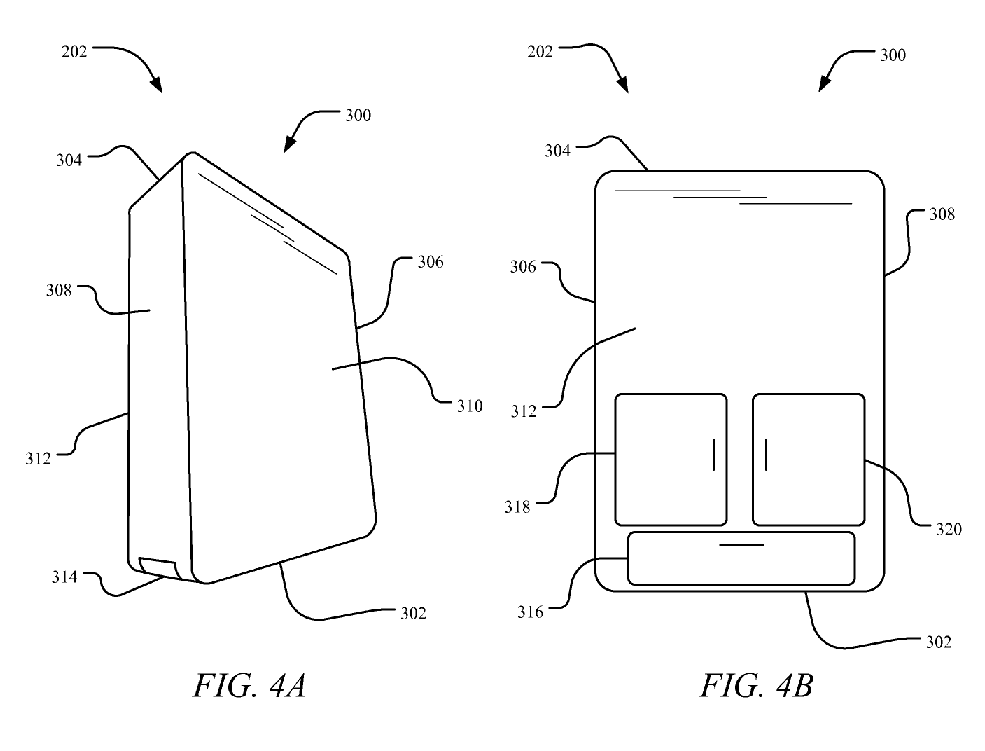

For a description of example internal components and air flow through the air purifier 202 in the form of a powered air purifying respirator, reference is made to FIGS. 4A-7. Turning to FIGS. 4A and 4B, a side perspective view and a back view of the air purifier 202 is shown. In one implementation, the air purifier 202 includes a housing 300 to enclose the internal components of the air purifier 202. For instance, the housing 300 may comprise a chassis housing with top wall 304, bottom wall 302, side walls 306 and 308, and a back wall 312. In one implementation, a front wall 310 is a removable cover which, when attached or affixed to the chassis housing encases the internal components of the air purifier 202.

In some implementations, one or more of the walls 302-312 may be configured with openings to provide access to internal components, provide for air flow into/out of the air purifier 202, and/or the like. For example, the top wall 304 may include an opening or other type of access port to allow for access and replacement of internal components (e.g., a primary filter module) and to allow for air flow out of the air purifier 202, as described herein. In one implementation, the bottom wall 302 includes an opening or other type of access port to allow for attachment/integration of an air entry mesh 314, and/or to allow for access and replacement of other internal components. The back wall 312 may include additional covers (e.g., covers 316-320) for accessing compartments holding internal components. For example, the cover 316 may be used to access a pre-filter, and the covers 318 and 320 may be used to access batteries. It will be appreciated, however, that more or fewer covers may be included for accessing a variety of different internal components.

Moreover, while the removable cover 310 illustrated in FIG. 4A extends the entire length of the chassis housing, the disclosure is not so limited. For instance, in certain implementations, the chassis housing may be enclosed by one or more cover portions that extend along portions of the chassis housing, for example, such that a first cover portion encloses a portion of the chassis housing comprising mechanical and electrical system components and a second cover portion encloses a portion of the chassis housing comprising the primary filter module.

The housing 300 may be a variety of shapes and sizes and may be constructed from a light-weight, durable material. By way of non-limiting example, suitable materials for construction of the housing 300 include anodized aluminum, titanium, titanium alloys, aluminum alloys, fibrecore stainless steel, carbon fiber, Kevlar™, polycarbonate, polyurethane, or any combination of the mentioned materials.

In one implementation, air enters into the air purifier 202 initially through the air entry mesh 314 attached or integrated at the bottom wall 302 of the housing 300. Although illustrated with the air entry mesh 314 disposed at the bottom of the housing 300, the disclosure is not so limited and alternative configuration and orientations are within the scope of the disclosure. For instance, the air entry mesh 314 may be configured on any of the other walls 304-312. In one implementation, the air entry mesh 314 is a separate component which is attached to the housing 300. In another implementation, the air entry mesh 314 is integrated into the housing 300 as a unitary component. The air entry mesh 314 may be constructed from a light-weight, durable material.

As described herein, the air entry mesh 314 provides initial protection against large particulates as well as offers a low resistance entrance for unfiltered air. As illustrated, the air entry mesh 314 may extend slightly up the side walls 306 and 308 to allow air to enter the air purifier 202 even if it is placed on a surface that would block the majority of the holes of the air entry mesh 314 located on the bottom wall 302.

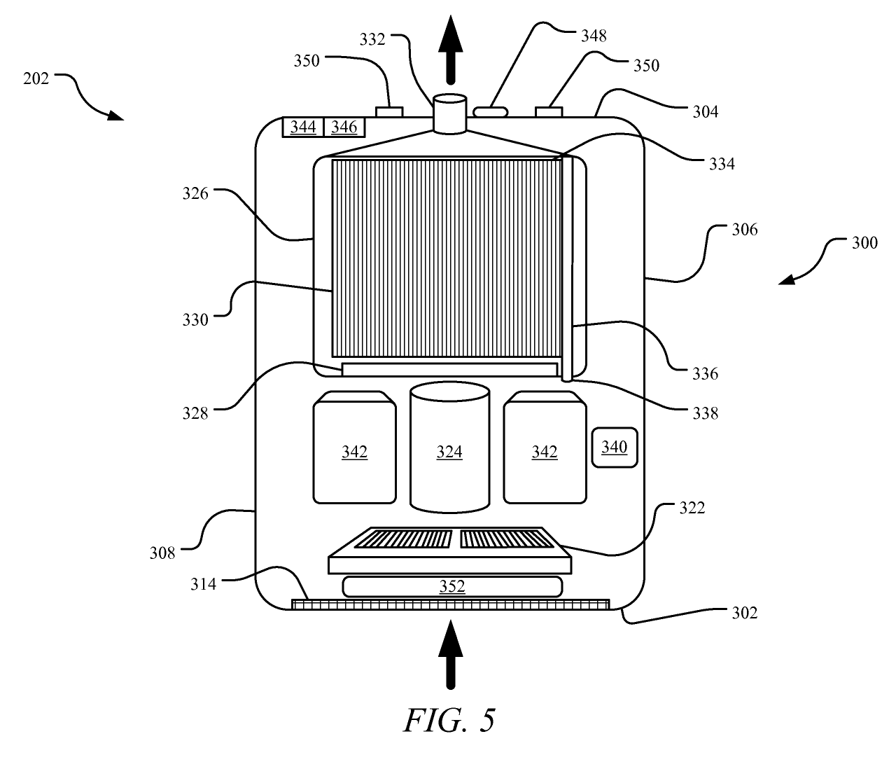

As can be understood from FIG. 5, in one implementation, the air entry mesh 314 serves as an initial entry port for non-filtered air to enter the respirator 104 and is therefore also the first region of large particle filtration. The openings of the air entry mesh 314 are sized and spaced such that each of the openings are large enough to reduce resistance to air being drawn into the air purifier 202 and small enough to prevent very large particles from entering the air purifier 202. In one implementation, the openings in the air entry mesh 314 are generally cylinders of a finite thickness and diameter arranged in parallel. The parallel arrangement of the openings allows for a linear reduction in flow resistance that is directly related to the number of openings without sacrificing the minimum opening dimension, which in turn governs the size of particles that are allowed to pass through the openings.

In one implementation, the air is pulled through the air entry mesh 314 into one or more fans 324. In another implementation, after entering the air purifier 202 through the air entry mesh 314, the air is drawn through one or more pre-filters 322 using the fans 324. The pre-filter 322 filters large particles that could potentially build up on and/or damage the fans 324 and/or a primary filter module 326, which would decrease the lifetime of primary filters 330 within the filter module 326.

The pre-filter 322 may have any suitable filter pore size and may be formed in pleated or non-pleated configurations. For example, the pore sizes of the pre-filter 322 can range from approximately 0.1 micron-900 microns. Such pore sizes, and pleating/non-pleating configuration generally produce very low pressure drop. The pre-filter 322 may be formed from a variety of suitable filter materials used in High-efficiency particulate arrestance (HEPA) class filters. For instance, the pre-filter 322 may be formed from Polytetrafluoroethylene (PTFE), Polyethylene terephthalate (PET), activated carbon, impregnated activated carbon, or any combination of the listed materials. These materials may also be, optionally, electrostatically charged. In one implementation, the pre-filter 322 is a single pleated or sheet of material. In another implementation, the pre-filter is co-pleated or laminated with other desired materials for combined benefits. By way of non-limited example, the pre-filter 322 may be configured as a 0.5 micron PET material co-pleated with activated carbon, potassium permanganate impregnated activated carbon material, and the like. In other implementations, the pre-filter 322 may include one or more hydrophobic layers, for example to minimize intrusion of moisture/water into the system. The hydrophobic layer(s) may be of generally large pore size (e.g., approximately 1 micron in diameter). By way of example, the PET material may provide filtration for particles 0.5 microns and up, the activated carbon may provide filtration of volatile organic compound (VOCs), smaller acid (SOx/NOx) gas molecules, and the like, as well as removal of odors/smells, and the hydrophobic layer may minimize intrusion of moisture/water.

The fans 324 are disposed near an air inlet 328 of the primary filter module 326. In one implementation, the fans 324 are disposed along the air path between the pre-filter 322 and the primary filter module 326. The fans 324 generate a positive pressure air flow that pulls air from outside through the air entry mesh 314 through the pre-filter 322 into the primary filter module 326 and out an air outlet port 332 through a filter module outlet 334. In one implementation, the one or more fans 324 operate at high hydrostatic pressures (e.g., 3-5 inches of water) and generate high flow rates up to 300 SLM. In certain implementations, to achieve high efficiency for the primary filter module 326, the fans 324 operate between approximately 50 and 300 SLM. The fans 324 may operate at various speeds, for example, low (100 SLM), medium (130 SLM), and high (180 SLM). There may be sound proofing material around the fans 324. The material may be, without limitation, silicone.

In one implementation, the one or more fans 324 includes a plurality of fans in a series stacked, axial fan configuration (stack). Without intending to be limited by theory, as opposed to a parallel configuration (i.e., both fans disposed beside each other), the series (stacked) configuration allows the pressure output to be additive, whereas a parallel configuration results in an increase in overall flow. In one implementation, the fans 324 provide over a 70,000 hour runtime.

The static pressure of the air purifier 202 may be increased by including a plurality of fans 324 in a stacked configuration having contra-rotating two stage axial impellers. In one implementation, two or more stacked fans 324 are provided, as described above, which rotate in opposite directions with the upstream fan having a pitch angle that is approximately 8-10 degrees higher than the fan further downstream.

The fans 324 direct the air into the primary filter module 326 through the air inlet 328. The primary filter module 326 may be configured to include one or more primary filters 330 and optional post-filter(s). In one implementation, the primary filters 330 are oriented parallel to the direction of air flow. In another implementation, the primary filters 330 are oriented at an angle relative to the direction of airflow. Other configurations and orientations are contemplated as well. In one implementation, the primary filter module 326 includes a pressure sensor intake port 338 and a pressure sensor intake 336 to measure the pressure within the primary filter module 326 during operation. The air purifier 202 may further include a pressure sensor chip 348 configured to send pressure readings from outside the air purifier 202 to be analyzed and recorded by a controller 340, which may be substantially similar to the controller 118.

As described herein, the air purifier 202 may include one or more pre-filters 322, primary filters 330, and post-filters. By way of non-limiting example, one or more optional charcoal post-filters, one or more optional charcoal pre-filters, and one or more primary filters 330, may be included. In certain aspects, the post-filters may be added to the system for increased protection, for example, from inhalation of VOCs, any outgassing that may occur from any of the filters 322 or 330 or glue used in the system, and the like. Any suitable filter material may be used as the pre-filters 322 and post-filter, including, by way of non-limiting example, activated carbon filter material that has been properly treated to prevent outgassing and fine particulate emission from the carbon filter itself. However, any suitable filter material may be used, and the disclosure is not limited to activated charcoal. Further, any suitable filter material may be used as the primary filter 330, including, but not limited to, a composite filter media.

For instance, by way of non-limiting example, the primary filters 330 may include any HEPA type membrane material, e.g., with a 0.1 micron-0.3 micron pore size made from an inert material such as PTFE, PET material, activated carbon, impregnated activated carbon, or any combination of the listed materials. These materials may also be, optionally, electrostatically charged. In one implementation, the primary filters 330 are a single pleated or sheet of material. In another implementation, the primary filters 330 are co-pleated or laminated with other desired materials for combined benefits. By way of non-limited example, the primary filters 330 may be a composite material including more than one layer of filter materials copleated using a thermal procedure (adhesiveless), or adhesive-based bonding to attach one or more additional layer(s) of filter material, load bearing material, activated carbon for added system protection, impregnated activated carbon, and/or the like. In one implementation, adhesive-based bonding is used, employing adhesives having low or no outgassing. Stated differently, the primary filters 330 may be formed by bonding, copleating, laminating or otherwise attaching additional layers to suitable filter materials.

In one particular implementation, the primary filter 330 includes an extra layer of Ultra-high-molecular-weight polyethylene (UHMWPE) added to the filter stack to increase the filter efficiency. The layers of the primary filter 330 may be affixed/bonded in any suitable manner, e.g., by thermal bonding, crimping, adhesive, etc. In certain implementations, the layers of the primary filter 330 may be bonded by crimping the edges and pleating together by loading into a collator. In other implementations, adhesive with a thickness range between approximately 0.5 oz per square yard to 3 oz per square yard, e.g., 1 oz per square yard may be used. Without intending to be limited by theory, the adhesive may add resistance to the primary filter 330, which may create and add pressure drop to the system. Thus, in one implementation, the UHMWPE membrane is formed as thin as possible. Alternatively, or in addition, any adhesive may be reduced or removed to decrease pressure drop and to reduce outgassing and VOCs emitted therefrom. If desired, activated carbon may also be added to remove VOCs (odors and chemical fumes).

In another particular implementation, the primary filter 330 includes a plurality of thermally attached layers, including a first PE/PET layer, an activated carbon layer, a first PTFE membrane layer, a second PE/PET layer, a second PTFE membrane layer, a third PE/PET layer, a second activated carbon layer, and a fourth PE/PET layer. The activated carbon layers remove VOCs.

In one implementation, the air purifier 202 provides a particle velocity at the surface of the primary filters 330 (face velocity) less than or equal to 5 cm/s, 4 cm/s, 3 cm/s, 2 cm/s, or 1 cm/s. At such face velocities, the collection efficiency for the primary filters 330 in the air purifier 202 is greater than 99.99%, 99.999%, 99.999%, 99.9999%, or 99.99999%, which greatly out performs conventional positive pressure respirators and filters. Further, using a face velocity of less than or equal to 5 cm/s, 4 cm/s, 3 cm/s, 2 cm/s, or 1 cm/s, also produces a lower pressure drop across the primary filters 330, as compared to using a higher face velocity, e.g., greater than 5 cm/s, which is beneficial for overall system efficiency (e.g., less demanding for the fans 324).

In one implementation, the air purifier 202 has a filter efficiency of 99.99999% down to 0.01 microns. The air purifier 202 utilizes composite filter media in combination with optimized flow rates, to provide highly cleaned air at a positive pressure to one or more users regardless of their pulmonary output or size. The air purifier 202 can deliver positive pressure air at flow rates of up to and greater than 300 SLM (standard liters per minute), 100-300 SLM, 100-200 SLM, etc. This permits users with large lung volumes to utilize the air purifier 202 at high exertion levels, making it a versatile platform that can be used in high pollution urban environments and in high particulate occupational areas.

As described herein, in addition to superior filtration efficiency, the air purifier 202 achieves reduced power consumption. Generally, the functionality of a filter over time has a direct effect on the performance and efficiency of a power source 342. For instance, as a filter is loaded with particles the overall resistance of the filter is increased. When the filter resistance increases, it requires more energy output from the power source 342 to drive the fans 324 at the flow rate/face velocity set in the unloaded state. As such, in some implementations, the respirator includes the pre-filters 322 to extend the life of the primary filter 330 and reduce power consumption. The power source 342 may utilize, without limitation, direct current (DC), alternating current (AC), solar power, battery power, and/or the like. In one particular implementation, the power source 342 includes one or more lithium ion batteries that are rechargeable with a DC 15V power adapter. The batteries in this case each have a run time of approximately 12.87 hours at 100 SLM, 8.36 hours at 130 SLM, and 4.5 hours at 180 SLM.

In one implementation, the controller 340 manages the power consumption of the air purifier 202 by controlling the charging and discharging of the one or more power sources 342. As described herein, the controller 340 receives an input from the user device 212 and/or controls on the air purifier 202 and in response, activates the one or more fans 324 for providing airflow through the air purifier 202 at various flow rates. In one implementation, the user device 212 communicates with the respirator 102 via a connection 346 (e.g., a wired connection or wireless connection). The controller 340 may also alter the speed of the fans 324 according to the charge level of the power sources 342 and may convert a provided input power through a power connector 344 to an appropriate charging voltage and current for the power sources 342. The controller 340 further communicates the with the monitor 102 via the connection 346 to monitor and/or manage operation of the air purifier 202 and air quality.

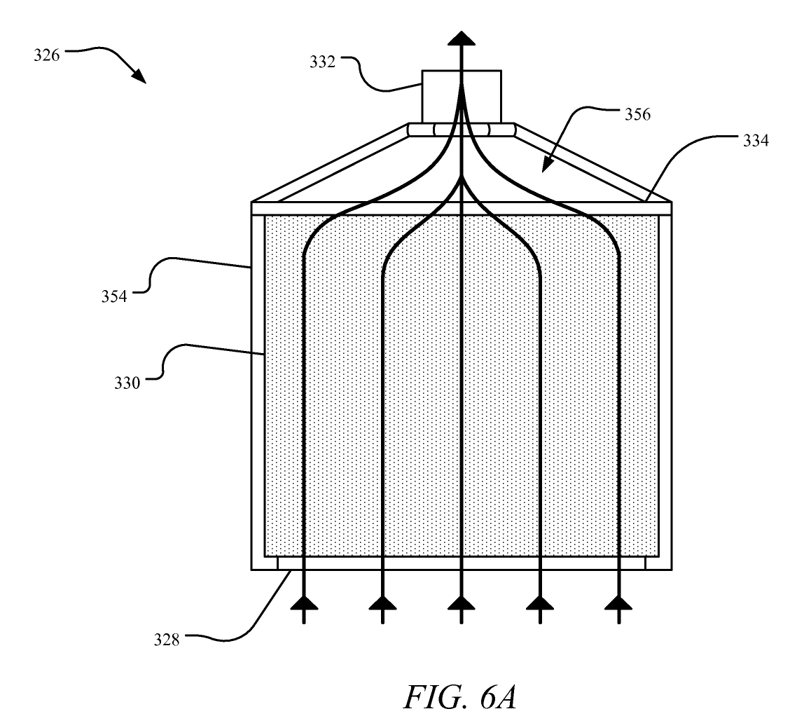

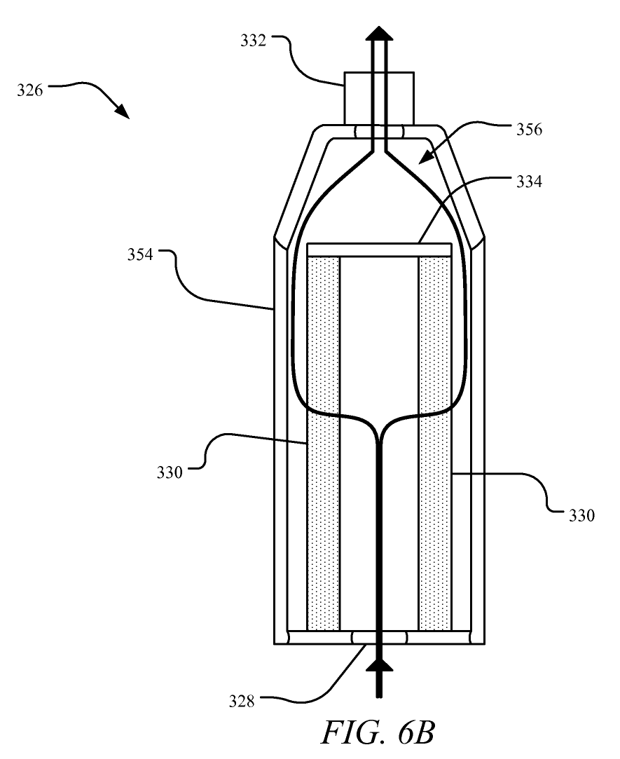

FIGS. 6A and 6B illustrate the air flow through the primary filter module 326. Upon entering primary filter module 326 through the air inlet 328, the air flow is directed along one or more paths through the primary filters 330 along a length of sides 354 of the primary filter module 326 through the filter module outlet 334. The filtered air combines in a purified air section 356 before being output through the air outlet 332.

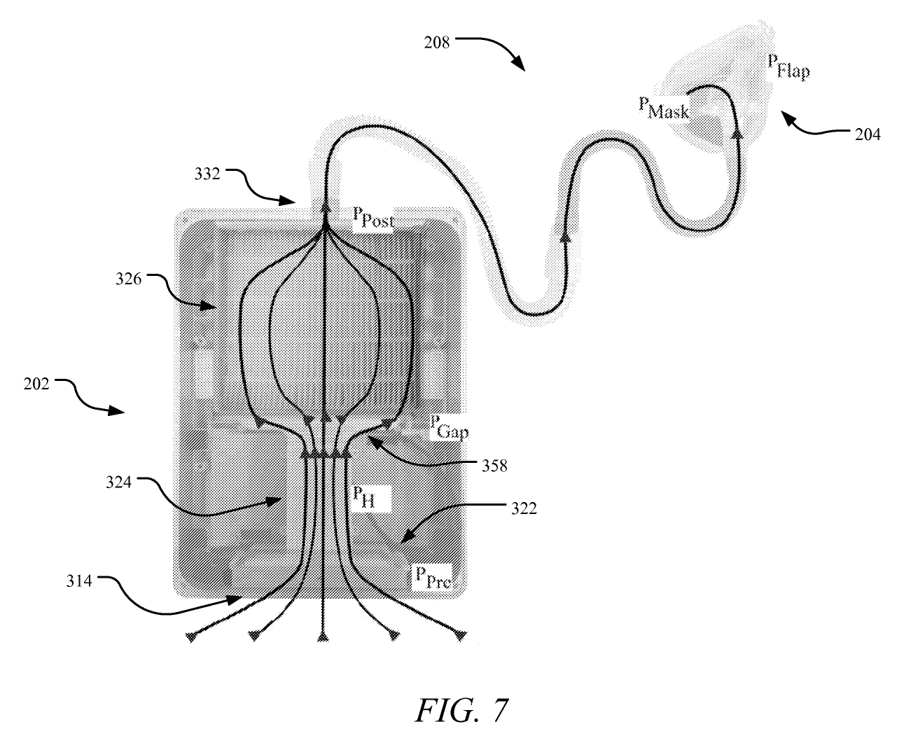

Turning to FIG. 7, an example hose 208 having a tapered diameter is shown. In one implementation, the hose 208 tapers in diameter proximally. Such a tapered configuration of the hose 208 may be secured though a carrying strap of a carrying case, such that the hose 208 remains secured inside the strap out of the way of the user. Moreover, the tapering provides a lower pressure drop through the air filtration system 104 as compared to a single, larger diameter hose.

A plurality of sensors may be located throughout the airflow path and in communication with the controller 340. In one implementation, the controller 340 receives the pressure readings and utilizes the readings to determine the pressure drop at various locations, including, without limitation, at the air entry mesh 314, the pre-filter 322, the primary filter module 326 (e.g., based on a gap 358 between the filters and the fans 324), the post-filter near the outlet 332, the hose 208, the mask 204, and a flapper valve within the mask 204. These regions can experience a press drop due to the geometric changes and restrictions.

In one implementation, the pressure drop for the entire air filtration system 104 is calculated using the following equation:

$${P}_{H} \geq {\sum }_{i}^{n} {P}_{i}$$

Here, P_{H} is the hydrostatic pressure output by the fans 324 and Pᵢ represents each aspect of the respirator 102 that could cause a pressure drop. For example, using the pressure readings from each of the components detailed above, the equation would be:

P_{H}≧P_{grate}+Pₚᵣₑ+P_{gap}+P_{filter}+Pₚₒₛₜ+P_{tube}+Pₘₐₛₖ+P_{flap}

The sum of each component's pressure drop must not exceed the total hydrostatic pressure that the fans 324 are capable of producing. In one implementation, the fans 324 are able to operate at 3 inches of water (IW) of pressure with a ceiling operating output of 4.8 IW. Further, in one implementation, the air purifier 202 operates at a normal flow rate of 100 standard liters per minute (SLM), with a maximum flow rate of 200 SLM.

In one implementation, a pressure drop across a filter (e.g., the pre-filter 322, the primary filter 330, the post-filter, etc.) may then be used to determine if the filter needs to be replaced. For example, as a filter nears the end of its lifespan, the airflow through the filter decreases, causing the pressure drop across the filter to decrease. Once the pressure drop has fallen below a threshold, the controller 340 may trigger an indicator alerting the user of the need to replace the filter. In another implementation, the air pressure data may be used in conjunction with usage data to better determine whether the filter needs to be changed.

The controller 118 may include the controller 340 and the various operations of the air purifier 202 described with respect to FIGS. 4A-7 may be controlled by the monitor 102 using the controller 340. Further, the sensors 116 may comprise the various sensors for detecting particles, measuring pressure, monitoring fan speed, and/or other operational parameters described with respect to FIGS. 4A-7, with the health and/or air filtration data captured using the sensors 116 being communicated to the monitor 102 for analysis via the controller 340.

In one implementation, one or more particle detectors 252 are configured to detect one or more, two or more, or three or more particle detection levels. For example, the particle detectors 252 may include three primary detection levels, such as >PM2.5, PM2.5, and PM10. The particle detectors 252 may utilize various techniques for detecting particles of various sizes, including, without limitation, laser particle counter, optical particle counter, TOF particle sizer, inertial classifier, low pressure microorifice impactor, and/or optical microscope. The controller 340 obtains the particle count and communicates it to the monitor 102 for analysis.

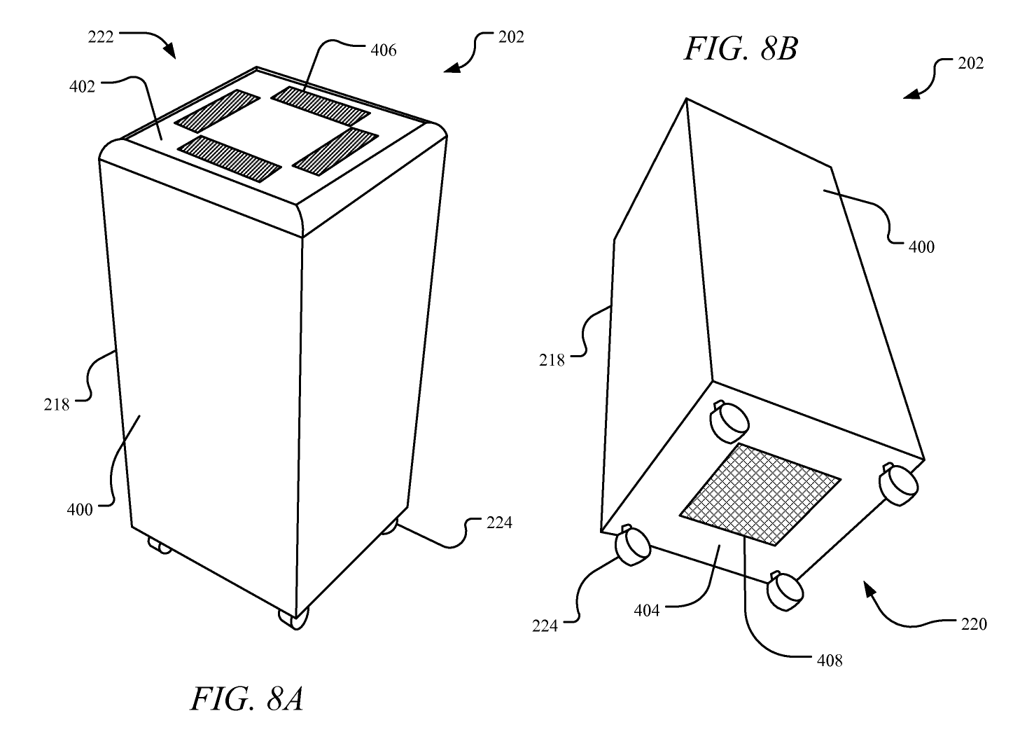

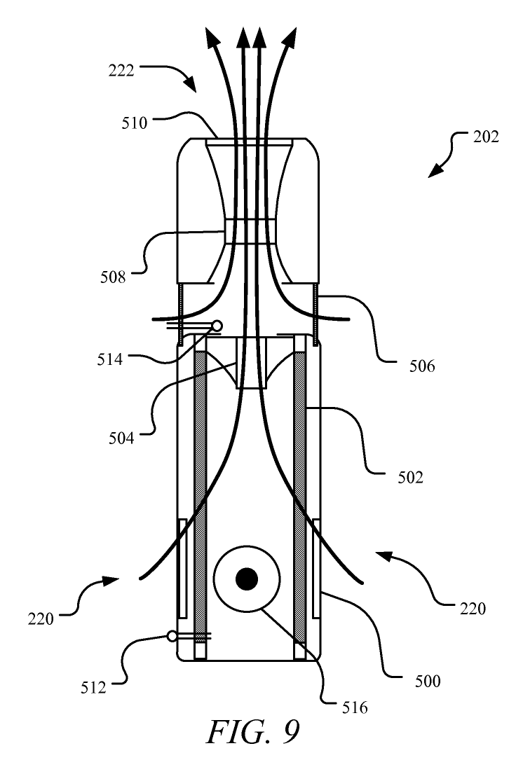

Turning to FIGS. 8A-9, a description of example internal components and air flow through the air purifier 202 in the form of a room air cleaner is provided. As shown in FIGS. 8A-B, in one implementation, ambient is drawn from the room into the housing 218 through the pre-filter 208 into a filter box 210 using one or more filter fans 212. In one implementation, the filter box 210 includes one or more surfaces 400 extending between a distal surface 404 and a proximal surface 402, with the filter fans disposed along an air path between the distal surface 404 and the pre-filter 408 and the proximal surface 402 positioned relative to vents 406.

Referring to FIG. 9, in one implementation, ambient air is drawn into the air purifier 202 through one or more inlet vents 500 disposed at the air inlet 220, which may be positioned anywhere on the housing 218 including, without limitation, the distal surface 204 or one or more of the side surfaces 200. The inlet vents 500 may include grating to filter large particulates. The air is drawn through the inlet vents 500 and directed at a primary filter 502 using one or more filter fans 504. Stated differently, the filter fans 504 generate a filtration air flow through the primary filter 502. The filter fans 504 may be oriented in a stacked configuration as detailed herein.

In one implementation, a recirculation air flow is generated by drawings air through recirculation air inlets 506 using one or more recirculation fans 508. The recirculation air inlets 506 may be protected by grates and may include one or more pre-filters 322, as described herein. Purified air is output through one or more outlet vents 510 at the air outlet 222. Thus, the air purifier 202 separates air filtration from air recirculation, thereby enhancing efficiency.

The air purifier 202 may include one or more differential pressure sensors (e.g., pressure sensors 512 and 514). In one implementation, the pressure sensor 512 measures pressure of a cavity of the housing 218 relative to the atmosphere. Thus, the pressure sensor 512 effectively measures any particle loading that could exist on the primary filter 502, which would cause an increase in the pressure differential between the cavity and the atmosphere. Once this pressure differential reaches and exceeds a predetermined pressure drop, in one implementation, an indicator LED on the controller 118 would illuminate, signaling that the primary filter 502 requires changing. Alternatively or additionally, the air purifier 202 may send an alert to the user device 212 or generate other alerts, including visual, audio, tactile, and/or the like.

The primary filter 502 may comprise a total area of 100-504 square feet (e.g., 100, 125, 150, 175, 200, 225, 250, 275, 504 square feet) with one or more layers of load bearing material and filter material. In one implementation, the primary filter 502 construction provides the air purifier 202 with an efficiency of 10-10 of particles down to 10 nanometers in size at a face velocity of 0.25 cm/s at a flow rate of 100 CFM. This efficiency allows the filter to capture UFPs and airborne viruses, preventing inhalation of dangerous particles by the users 116. The large surface area of the primary filter 306 filters nanoparticles, such as viruses, smoke, cat dander, and other allergens, with a collection efficiency better than 99.99999% for 30 nm size particles. With such a collection efficiency, a carbon activated filter, which is pressure drop intensive, is unnecessary for fine particle removal.

More particularly, because the size of the primary filter 502 is large filter, the face velocity is very small. In one implementation, the air purifier 202 runs at 200 CFM, which is 5663 l/min standard liters per minute (SLM). In the United States, the room size rating of a purifier using 200 CFM is 404 ft2 (27.9 m2). This rating means that there will be 5 air changes per hour (ACH) in a 404 ft2 (27.9 m2) size room. The flow rate of 200 CFM (5663 SLM) equates to a filter face velocity of approximately 1.2 cm/s. This face velocity is very slow, increasing the collection efficiency. Another advantage to using a larger size is the pressure drop on the primary filter 502 is very small. Running the air purifier 202 at 200 CFM will only have a pressure drop of 0.18 in (0.47 cm) across the primary filter 520, permitting slower fan speeds and reducing noise level and power consumption.

In one implementation, the filtration fans 504 draw air through the primary filters 502 through the interior of the housing 218 and through the outlet vents 510, as shown in FIG. 9. The filtration fans 504 may include any suitable fan configuration as described herein. For example, the filtration fans 504 may be configured to generate a static pressure of 2.4 inches of water at a max flow rate of 500 CFM with a particle face velocity of 1 cm/s. In one implementation, the filtration fans 504 include one fan to move adequate air to filter a given volume of air. In another implementation, the filtration fans 504 include a plurality of fans placed in series to increase the overall static head pressure in the air purifier 202.

In one implementation, the pressure sensor 514 is disposed on the inside of the housing 218 to generally serve as a control. The pressure sensor 514 may be configured to monitor a head pressure and control the filtration fans 504. For example, the sensor may regulate the power to the filtration fans 504 to maintain a flow rate set by the controller 118 and/or the user device 212. In one implementation, the pressure sensor 514 is set at 0.3 inches of water.

The recirculation fan 508 may be a high flow fan disposed near the air outlet 222 to draw ambient air from the room and circulate all the air, thereby directing unfiltered air at the air inlet 220. In one particular non-limiting example, the recirculation fan 508 has a max flow generation of approximately 600 CFM. The outlet vents 510 may include grating to prevent debris from falling into the air purifier 202, as well as prevent any children from putting their hands into the air purifier 202 and injuring themselves with the recirculation fan 508.

In one implementation, the primary filter 502 includes a high surface area (e.g., 100-504 square feet) of filter membrane material, enabling operation at 500 CFM with a face velocity of 1 cm/s, thereby achieving filter efficiencies of 99.9999%. In some cases, a single particle may be sufficient to cause infection. In one implementation, the air purifier 202 is thus configured to remove all particles from a room. As an example, consider a large room that has a volume of 1152 cubic feet that contains that contains a virus particles (say the influenza) at a concentration of 16,000 per cubic meter—at this concentration the total number of influenza particles in the room would total approximately 522,153. Using the air purifier 202, only 0.53 or approximately 1 particle would remain the room. When the fan speed is switched to the lower level of 100 CFM, the air purifier 202 would remove all of the particles from the room.

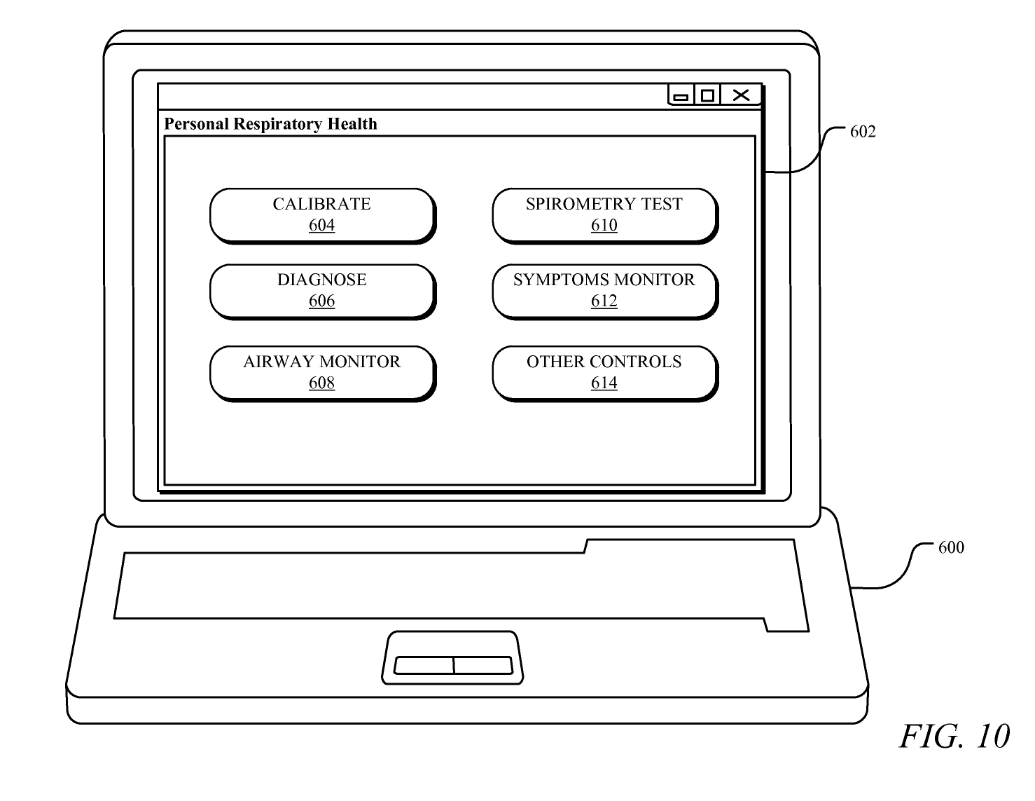

FIGS. 10-12 show example user interfaces generated by the monitor 102 and displayed in a browser window of a user device 600 (e.g., the user device 212, including, the consumer device 108, the administrator device 110, etc.) through which access to and interactions with the air filtration systems 104 and related data are provided. It will be appreciated by those skilled in the art that such depictions are exemplary only and not intended to be limiting.

Turning first to FIG. 10, in one implementation, the monitor 102 generates a personal respiratory health user interface 602 for accessing health monitoring analytics and/or feedback. In one implementation, the interface 602 includes calibration and air flow analytics 604, diagnosis analytics 606, airway monitoring analytics 608, spirometry test analytics 610, symptoms monitoring analytics 612, and other controls analytics 614, which may pertain to other aspects of the use, operation, and effect of the air filtration system 104.

In one implementation, the calibration and air flow analytics 604 may indicate a pressure response of the sensor 116 in response to a consumer's breath, and the monitor 102 may adjust the air flow rate of the air filtration system 104 accordingly as feedback. The monitor 102 may adjust the air flow rate by varying the duty cycle to compensate for the consumer's sensed breathing rate. In one implementation, calibration and air flow analytics 604 provide maximum and minimum air flow settings and/or prompt an initial calibration measuring the consumer's breathing while at rest and during heavy activity.

The diagnosis analytics 606 may include, without limitation, asthma diagnosis analytics, COPD diagnosis analytics, and/or diagnosis analytics for other medical conditions. In one implementation, the monitor 102 receives input regarding consumer information, including, but not limited to race, age, sex, height, weight, and/or symptom information. The monitor 102 uses the input to generate the diagnosis analytics 606 including race specific “normal lung function” using a linear regression technique and an analysis of consumer lung function with respect to the normal lung function.

The airway monitor analytics 608 includes analytics regarding a condition of the consumer airway, for example, in the context of asthma, COPD, or similar diagnoses. In one implementation, the airway monitor analytics 608 provides real time monitoring of a consumer's measured airways resistance. By way of example, the monitor 102 may measure airway resistance using a ventilator or a lethysmography box. The monitor 102 calculates airway resistance using the following expression:

$$R = \frac{Δ P}{Q}$$

where R is the airway resistance, ΔP is the pressure difference generated by the user from breathing, and Q is the flowrate. The airway resistance changes with breathing effort, tidal volume, air quality, and/or the like. The airway monitor analytics 608 identifies any change in airway resistance for a consumer and may generate feedback in response.

In one implementation, the airway monitor analytics 608 includes the airway resistance calculated breath by breath using a difference between the most negative inspiratory pressure during a breathing cycle and the most positive pressure right before the next inhalation using the volumetric flow rate at 0.5 seconds. The monitor 102 averages the calculated airway resistance over an averaging time frame, for example, between 1 and 15 minutes (e.g., approximately 10 minutes), to identify any changes. To eliminate major outliers from the analysis, the monitor 102 may have boundary conditions to exclude events such as coughs and sneezes that can be identified by sharp increases (spikes) in pressure value over a short period of time (e.g., 0.5-3 seconds). Another method to improving the data analysis may include increasing the averaging time frame (e.g., up to 1 hour). The airway monitor analytics 608 may indicate a significant change in airway resistance where the change is in excess of a percentage threshold, such as 10-20%.

Where the airway monitor analytics 608 indicates that airway resistance has increased, the monitor 102 may generate feedback in the form of a questionnaire displayed on the interface 602 to validate the symptoms of asthma (e.g., an asthma control test). The monitor 102 may generate further feedback based on the results of the questionnaire, including, for example, suggestions on how to proceed if it is determined that they are suffering from asthma, COPD, or other airway restrictive triggered ailment. Other feedback may include alerting a healthcare or emergency service professional via the administrator device 110 depending on a severity of the airway monitor analytics 608.

In one implementation, the spirometry test analytics 610 include the results of a spirometry test performed by switching the fan of the air filtration system 104 off or to a low setting and prompting the consumer perform spirometry maneuvers. From these maneuvers, the monitor 102 analyzes the generated flow response curves and determines relevant pulmonary values such as FEV1.

The symptoms monitoring analytics 612 may include respiratory monitoring analytics including normal breathing patterns, involuntary and voluntary breathing for the consumer, and/or the like. The monitor 102 may receive an activity level (i.e. exercising, resting, walking, etc.) for the consumer and may determine the consumer's breathing pattern for these activities. The monitor 102 may further track coughing and sneezing as outlier data points in the symptoms monitoring analytics 612. This symptoms monitoring analytics 612 coupled with temperature sensed by the sensors 116 may be used to indicate the health status of the consumer, including whether the consumer has a cold or the flu. As described herein, the monitor 102 may further obtain a heart rate for the consumer to monitor cardiovascular and respiratory performance of the consumer during varying activity levels and to provide a more accurate measure of various health symptoms and conditions.

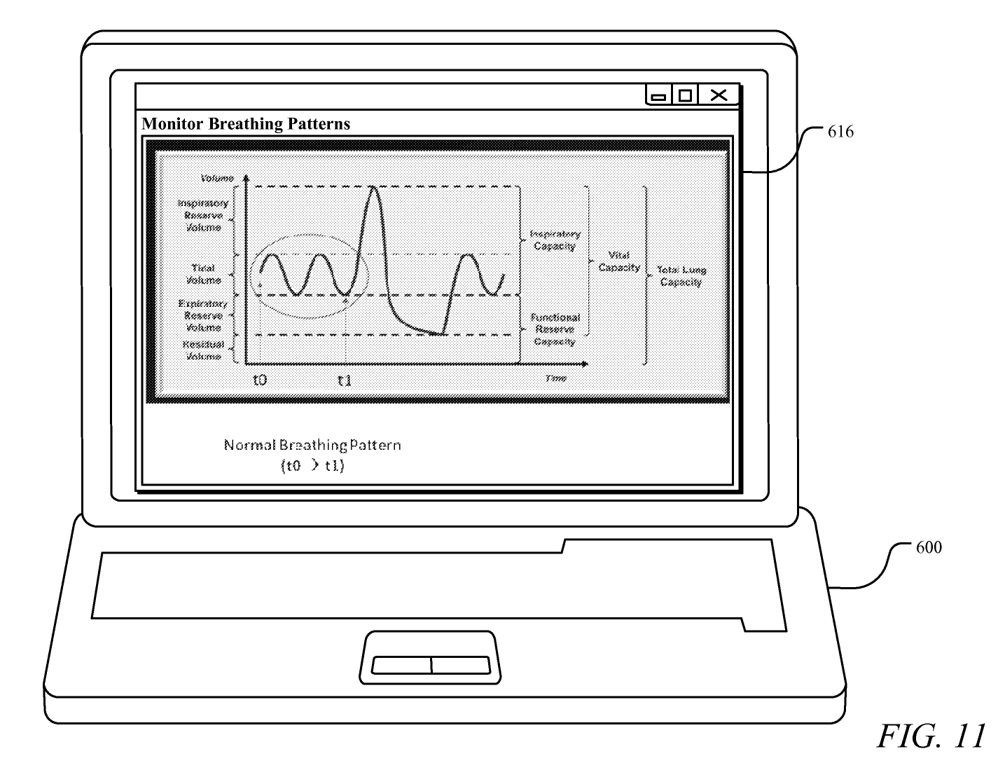

As can be understood from FIG. 11, which is an example respiratory health user interface 616 for monitoring breathing patterns, in one implementation, health data collected from the sensors 116, including pressure sensors, may be used to directly monitor or indirectly infer breathing patterns of the consumer. In one implementation, the health data can be used to measure FEV1. Normal breathing is relative to the consumer's baseline activity level and as a result there may be multiple “normal breathing” settings based on activity. Nonetheless, once the baseline “normal breathing” pattern is established, abnormalities in breathing pattern from the baseline can be used to differentiate between healthy and unhealthy conditions of the consumer. Without being limited, normal breathing patterns may have a sinusoidal like pattern, as shown in the interface 616.

The example shown in FIG. 11 highlights an example of lung volume response to normal breathing. Since the monitor 102 measures pressure instead of volume over time the shape of the response may be slightly different with the overall sinusoidal pattern consistent. This is due to the fact that pressure and volume (for an approximately ideal gas) are inversely related due to the ideal gas law P=nRT/V where P is air pressure, n is number of mols, R is the gas constant, and T is the temperature.

When the normal breathing pattern is monitored in a controlled way, the monitor 102 establishes basic values for the consumer. Basic values are the measurements taken from the pressure/volume vs. time curves generated from the sensor. The type of values recorded from these plots may be frequency, peak-peak amplitude, RMS amplitude, and wavelength. These measurements work well for steady involuntary breathing patterns, however, real human breathing patterns are more complex since breathing is both voluntary and involuntary. When data is collected over a sufficient time period and the statistical power of the normal breathing curve is established involuntary breathing responses can be easily distinguished from voluntary breathing responses.

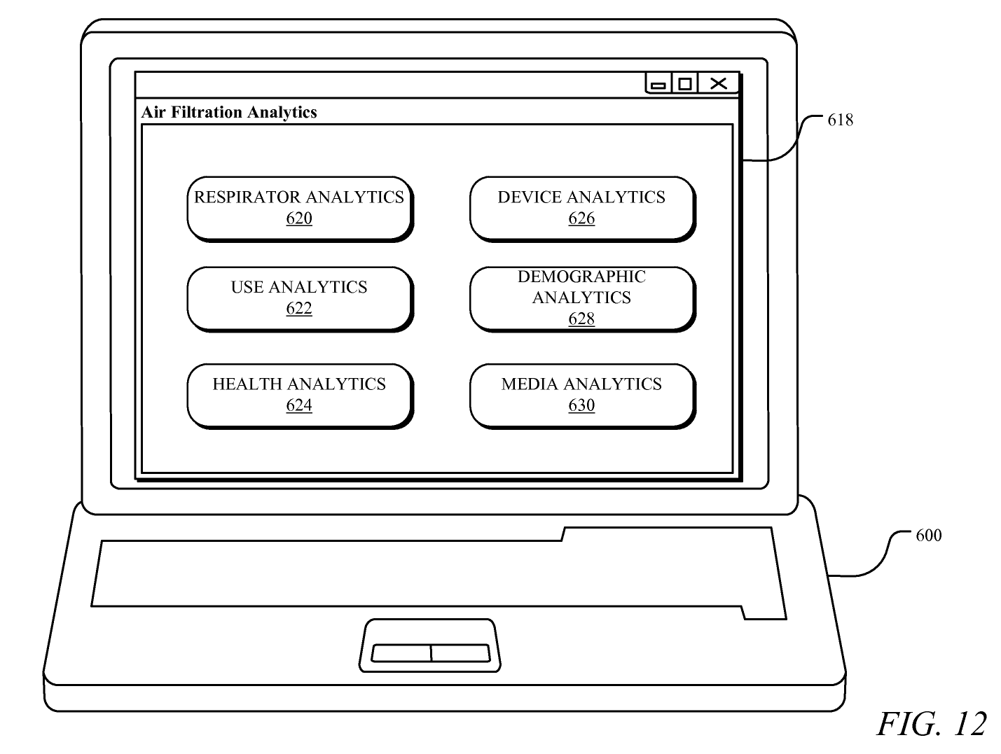

FIG. 12 is an example air filtration analytics user interface 618 generated by the monitor 102 and displaying air filtration analytics, including, without limitation, respirator analytics 620, use analytics 622, health analytics 624, device analytics 626, demographic analytics 628, and media analytics 630.

In one implementation, the respirator analytics 620 includes analytics relating to the air filtration systems 104 including operational data, such as power supply levels, charging time, fan speed and use, pressure within the air filtration systems 104, and/or the like. The power supply level may include data corresponding to the amount of power supply remaining. The power supply levels may be recorded during use and/or charging. The charging time may include data corresponding to a length of time of charging and an occurrence of changing. Stated differently, the charging time may indicate how long consumers charge the air filtration systems 104 and what time of day consumers charge the air filtration systems 104. This data may be used to determine when and for how long the air filtration systems 104 are being charged and determine a capacity of the power source. The charging time may further include data on an amount of power used for parasitic charging, for example, to charge the consumer device 108. The fan speed and use indicates when air filtration systems 104 is filtering and moving air into or through the enclosed space, such as the mask 208 and/or a room, as well as to determine the speed at which the fan is moving the air through the air filtration systems 104. The pressure corresponds to pre and post pressure measured within the air filtration systems 104, which may be used by the monitor 102 to assess the air filtration systems 104 operation and/or provide information regarding the consumer's pulmonary output.

Turning to the use analytics 622, in one implementation, the monitor 102 provides consumer use patterns, product use research, use compliance, extended use, and/or other use analytics. Consumer use patterns may include a location of the air filtration systems 104, a day and time of use of the air filtration systems 104, and/or spatial temporal geolocation use patterns of the air filtration systems 104, as well as timing and effectiveness during such use.

The use analytics 622 may further include predictions regarding use, effectiveness, and/or consumer or operational health. In one implementation, the use analytics 622 includes power source life predictions, pressure changes, fan life predictions, acute medical condition predictions, pollution explore predictions, pulmonary health snapshots, load and fatigue predictions, and/or the like.

The power source life predictions may include analytics generated based on use patterns to predict power source life length, failure, and/or recharging times. Additionally, data about battery operation under environments and filtration loads can be assessed. Fan life predictions and blower conditions may be similarly monitored and predicted.

The pressure changes may include a pressure differential between two points and/or a post-filter pressure. The pressure differential may be used to determine the resistance offered by the filter of the air filtration system 104 and be used to determine filter lifecycle and efficiency under a variety of operating conditions. The post-filter pressure may be used to assess when a consumer's pulmonary output is causing air to be forced back toward the filter of the air filtration system 104 and not out the exhaust. The monitor 102 may monitor the use analytics 622 for a sudden pressure change to indicate a potential problem.

The acute medical condition predictions of the use analytics 622 may predict an asthma attack. In one implementation, the monitor 102 measures a change in NO levels expelled from the mask 208, which is a precursor to an asthma attack. The monitor 102 calculates a likelihood of an asthma attack occurring and its severity in a within finite period of time for output with the use analytics 622 or as an alert. In some implementations, increased levels of expelled NO is generally associated with exposure to air pollution. Thus, the monitor 102 may provide an changes in NO in the use analytics 622 as a proxy for exposure to air pollution in non-smokers not predisposed to asthma.

The use analytics 622 may provide a pulmonary health snapshot, load and fatigue predictions, as well as other consumer use analytics. In one implementation, by knowing the heart rate and various expelled gases, the monitor 102 generates a pulmonary health snapshot including any changes in pulmonary health. Similarly, using body weight, heart rate, carbon dioxide production (VCO2), and oxygen consumption (VO2), the monitor 102 computes a respiration exchange ratio (RER) to provide analytics on consumer load and fatigue, which may be monitored over time for changes.

In one implementation, the use analytics 622 includes product use research, including feature use to determine an importance, desirability, and/or value of a feature based on consumer use of the feature of the air filtration systems 104 and/or the monitor 102. For example, the use analytics 622 may indicate: whether a CO sensor changes usage patterns of consumers or encourages consumers to purchase the air filtration systems 104; whether consumers use the air filtration systems 104 for parasitic charging of user devices, such as the consumer device 108; whether any features encourage or increase use; and/or the like. In one implementation, the use analytics 622 provide suggestions for experiments to determine importance, desirability, and/or value of a feature.

The use analytics 622 may further include extended use encouragement. For example, the use analytics 622 may indicate or predict when the filter, battery, fan, or other component of the air filtration systems 104 needs to be changed or replaced. In one implementation, the use analytics 622 proactively submits reminders to order a replacement part or automatically orders such parts. The use analytics 622 may include promotions to provide rewards to consumers for purchasing replacement parts. The monitor 102 may provide the use analytics 622 to various responsible parties, including, for example, a parent, healthcare provider, insurance company, and/or the like to monitor extended use and/or effectiveness.

In one implementation, the use analytics 622 is used to ensure personal heath and use compliance. The use analytics 622 may provide data on whether and how often a consumer is using the air filtration system 104, including, for example, fan speed and usage data, pulmonary ventilation data, to ensure that consumers are using the air filtration system 104 as recommended. Thus, rather than relying on self-report measures of compliance in occupational or industrial settings, behavioral indicators from the respirator can be used to determine compliance and shared with responsible parties.

The use analytics 622 ensuring personal health and use compliance may be used to monitor lung health of the consumer. In some implementations, the use analytics 622 indicates changes in respiration rates and exhalation volume, which will provide insight into pulmonary health. This can be accomplished through analyzing baseline pulmonary ventilation and pulmonary ventilation variability. Pulmonary ventilation may be tracked and compared with hearth rate data to establish baseline pulmonary output values to which changes can be assessed. If changes in pulmonary output exceed a specific threshold, the use analytics 622 may generate an alert. If pulmonary output has significant variability, this could be indicative of potential short-term and/or long-term health issues. Short-term changes in lung function, such as those brought on by an asthma attack, COPD, change in pollution, or other extraneous events, may be included in the use analytics 622, as well as long-term changes in lung function, both on the positive and negative end.

In one implementation, the use analytics 622 includes usage patterns indicative of health compliance. The short-term and long-term changes and pulmonary variability can also be used to assess specific health issues of users. For example, a sudden elevation in NO exhaled together with changes in exhalation volume can signal an oncoming asthma attack. When these changes are detected, the monitor 102 may send an alert to the consumer, administrator, or another responsible party, via a text or other communications medium so the necessary steps to prepare or prevent an asthma attack may be taken. A number and intensity of sneezes and coughs can be monitored and a sudden increase in these events could signal a change in consumer health. These increases could indicate overexposure to air pollutants, so the use analytics 622 may prompt additional use of the air filtration system 104. These increases could also be coupled with body temperature to determine the potential onset of an illness. When these increases are detected, the monitor 102 may send an alert to the consumer, administrator, or another responsible party, via a text or other communications medium so the necessary steps may be taken.

On the other hand, a long-term increase in NO exhalation may be a sign of exposure to air borne pollution. The use analytics 622 may include spatial temporal nature of these elevated long-term changes in NO exhalation to assist users in determining the source of the potential airborne pollutants. The monitor 102 may generate alerts regarding potential exposure to harmful air pollutants. Long-term sneezing and coughing could be a sign of chronic lung damage, so use analytics 622 may include information regarding lung health based on tracked sneezing and coughing Long-term Delta and pulmonary output variability can be examined and overlaid with usage and compliance data to determine an effectiveness of the air filtration system 104 in improving consumer health.

The use analytics 622 may further detail pollution exposure levels within a region having one or more of the air filtration systems 104, regional sneezing and coughs indicative of regional allergies, pollutants, or illnesses, an impact of air quality on health within a region, and/or the like. Short- and long-term deltas can also be combined with usage data to determine if consumers are using the air filtration system 104 and in situations or at times as recommended. In addition to short and long-term Delta, VCO2, heart rate, and respiration exchange ratio, which is an indicator caloric consumption, can monitored and changes in these values can signal changes in overall health or user fatigue. In addition to changes to user's pulmonary output, the ambient environment surrounding the consumer can also be monitored to provide alerts to changes that could be detrimental to health. Similarly, if CO levels rise beyond that which is safe, the user analytics 622 may include an alert to one or more parties.

In one implementation, the use analytics 622 provide industrial and occupational use compliance information based on industry requirements or thresholds. In one implementation, the use analytics 622 monitors the air filtration systems 104 within a company or industry to provide global reminders regarding part replacement, use compliance, and/or the like. The use analytics 622 may further monitor consumers as a group, for example, within a company or industry based on group thresholds. For example, the use analytics 622 may include information regarding pulmonary ventilation, long-term and short-term deltas, respiration exchange ratio, heart rate, body temperature, etc. as compared to industry thresholds. Using a proximal detection sensor via a peer-to-peer mesh network, the monitor 102 may alert others they should be using the air filtration system 104 based on the use analytics 622.

In occupational safety settings the use of safety equipment and engaging in safety practices is paramount. Thus, in one implementation, the monitor 102 provides the use analytics 622 to the administrator device 110 or other central authority to monitor use compliance by a group. Further, lung health of a group in an occupational or industry setting may be monitored using the use analytics 622. Similar to monitoring the pulmonary output of consumers using the air filtration systems 104 in noncommercial applications, pulmonary output (i.e., respiration rate, exhalation volume, NO and CO2 expulsion, and respiration exchange ratio) may be monitored with a focus on occupational use where hazards are identifiable and part of the job.

In the occupational and industrial use setting, the administrator device 110 may be used by various responsible parties to obtain the use analytics 622. Such responsible parties may include, without limitation, supervisors who may need to monitor compliance for rules or to boost workforce effectiveness, medical personnel who might need to monitor usage in response to a communicable disease outbreak, regulatory agencies who could use the information to ensure that safety regulations are being followed, insurance or other companies who will could monitor compliance data to make determinations about insurance rates, and/or the like.

In one implementation, the health analytics 624 includes environmental health and user health analytics pertaining to, without limitation, pulmonary ventilation, irregular breathing, carbon monoxide (CO), carbon dioxide (CO2) expelled, environmental safety (a presence of any contaminants in the environment of the consumer), oxygen consumption (VO2), nitric oxide (NO) expelled, heart rate, body temperature, and/or the like.

The device analytics 626 may include connecting device operation and product performance. In one implementation, the device analytics 626 identifies any sharable data from connected devices, such as the consumer device 108, the administrator device 110, the databases 112, and/or other devices connected to the monitor 102 and/or the air filtration system 104 via a wired or wireless connection. The device analytics 626 may include data pertaining to such connections, including whether the connection is wired or wireless, as well as data regarding or obtained from a mesh network of the air filtration systems 104.

In one implementation, the product performance analytics provided by the device analytics 626 includes performance quality data, feature performance data, and feature prediction data. The performance quality data may include operational, consumer use pattern, and environmental and customer health data. The device analytics 626 may use the performance quality data to identify and monitor quality issues in current products and generate recommendations for addressing quality issues or otherwise improving products. For example, battery, filter, and fan levels can be monitored and that information can be overlaid with temporal and geospatial data as well and pulmonary ventilation data to determine how the air filtration systems 104 are currently behaving and how the air filtration systems 104 may behave at different locations, during different times of the day, and at different intensities of usage. Such device analytics 626 would establish an empirically-derived baseline and define the parameters of specific operating conditions and product life-cycles.

Analysis of operational, consumer use pattern, environmental and customer health, and temporal geo-spatial data may be included in the device analytics 626 to generate forecasting models that will predict overall and specific component performance of the air filtration systems 104. For example, the device analytics 626 may be used, without limitation, to: determine and/or predict when specific components are or will be operating at a sub-optimal levels; alert users immediately when or before the air filtration system 104 begins operating at a sub-optimal level and proactively offer to remedy the potential issue; identify whether the product issues are related to specific manufacturing locations or suppliers to identify product defects before they occur on a large scale and require recalls.

The feature performance data and feature prediction data of the device data 626 may be used to: determine adjustments in the design to reflect what is needed based on real instead of theorized usage patterns and/or extrapolate consumer use patterns and predict what new features might be popular and useful to guide product development in order to build the product that will optimize consumer use, cost, data collection and overall benefit to the company and society.

In one implementation, the demographics analytics 628 includes demographic and psychographic data as well as inferential consumer metrics. The demographic and psychographic data may include basic demographic data at the time of sale of the air filtration system 104, such as age, gender, height, weight, overall health, location, occupation, or income inference data, reasons for device purchase, attitudes regarding air pollution, and initial impressions of the device and its features. The inferential consumer metrics may include data corresponding to system size, mask size and use patterns to enrich the demographic data and track the air filtration system 104 across various consumers and types of consumers.

The media analytics 630 may correspond to social media exposure, such as discussion of the air filtration systems 104 in social media, marketing, and/or social media sharing. In one implementation, the media analytics 630 includes data-driven persuasive marketing strategies for both current and potential consumers. For example, the media analytics 630 may generate marketing regarding usage over time in the context of publicly available air pollution data. For consumers whose usage data suggests use of the air filtration system 104 when air pollutants were elevated, a message validating the health benefits and/or providing rewards to encourage additional use may be included in the media analytics 630. For consumers whose usage data suggests a failure to use the air filtration system 104 when air pollutants were elevated, a message explaining the health benefits and consequences of failed use, along with rewards to encourage future use, may be included in the media analytics 630. The health benefits information may be presented in a form facilitating understanding by consumers, for example, equating pollution levels to cigarette intake, an aging of lung capacity, life expectancy reduction, and/or the like. The rewards may be provided in the context of a reward system where the consumers may earn points, which can be redeemed for discounts or other benefits.

The media analytics 630 may further include data identifying current and future consumer types. For example, the media analytics 630 may determine that a set of current consumers are athletes, with a different and perhaps heavier usage pattern than non-athletes. The media analytics 630 identifies these consumers and provides suggestions for building a product tailored to the consumers, as well as a marketing strategy for reaching other similar consumers. The media analytics 630 may further include marketing strategies or messages for sharing via social media to explain health benefits of the air filtration systems 104 and provide information related thereto.

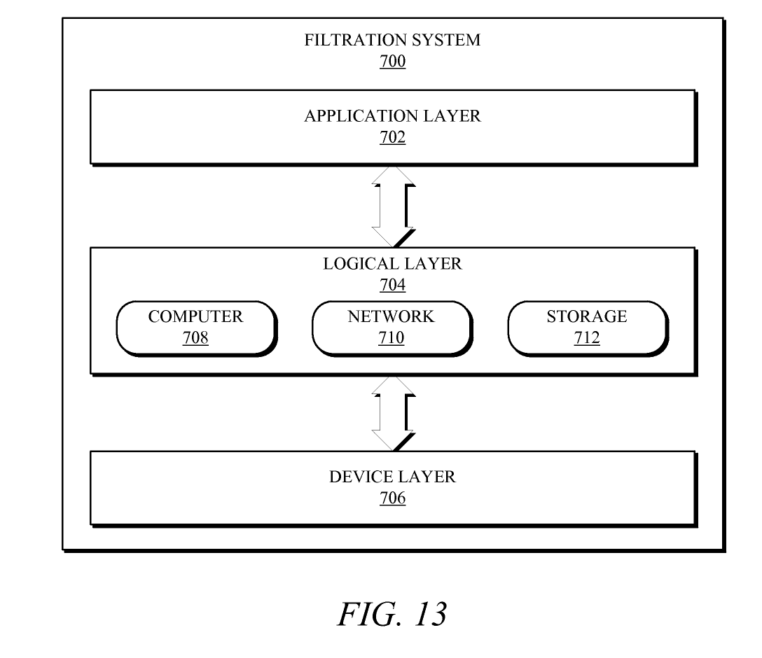

Referring to FIG. 13, a block diagram of an example air filtration system 700 is shown. The air filtration system 700 may be applicable to the air filtration systems 104 for capturing health data and/or air filtration data and generating analytics related thereto. In one implementation, the air filtration system 700 includes an application layer 702, a logical layer 704, and a device layer 706.

In one implementation, the device layer 706 includes the sensors 116, as well as other physical components of the air purifiers 202 and/or the air filtration systems 104 discussed herein, to purify air and capture health data and/or air filtration data. For example, the device layer 706 may include, among other components, an air entry, one or more pre-filters, one or more power sources, one or more blower/fan, a primary filter module including one or more primary filters and one or more optional post-filters, a controller 118, and various sensors 116 for monitoring operation of the air filtration system 104 and for detecting air particles, pollutants, contaminants, NOx, COx, and/or the like. In some implementations, the device layer 706 may include the hose 208, the mask 204, and/or other components of the air purifiers 202 of FIGS. 2-9.

The logical layer 704 may include various computer units 708, network units 710, storage units 712, and/or other computing units, as described herein. The air filtration system 700 may further include various logical software components in the application layer 702, which when executed generate, store, and/or communicate health and/or air filtration data.

In an example implementation, the filtration system 700 may include the one or more sensors 116 in the device layer 706 for monitoring operation and for detecting air particles, pollutants, contaminants, NOx, COx, and/or the like. In certain aspects, the sensor 116 may be located in a region of the filtration system 700 exposed to unfiltered air, a region of the filtration system 700 exposed to filtered air, or both a region of the filtration system 700 exposed to unfiltered and a region of the filtration system 700 exposed to filter air. The sensors 116 may include any suitable sensor and/or detector, depending on the parameter to be monitored, such as a fine particle sensor (e.g., particle detector 352), NOx, COx, and/or the like. Fine particle sensors which may be used in the context of the filtration system 700 include, without limitation: Shinyei PPD42NS model PM1 sensor, Shinyei AES-1 PM0.3 sensor, Shinyei AES-4 multichannel, SYHITECH DSM501A, NIDS PSX-01E, or the Sharp GP2Y1010AU0F. These sensors all operate in a similar fashion employing an optical scattering technique. However, other particle detection techniques, as discussed herein, may be utilized.

In certain implementations, the fan speed may be automatically adjusted by the logical layer 704 (e.g., the controller 118) based on measurements obtained by the sensor 116. These adjustments may occur when the filtration system 700 is operating in an “automatic” mode. By way of example, if the quality of the unfiltered air is detected to meet a minimum quality threshold, the controller 118 may slow the blower/fan to save energy. In certain embodiments, the filtration system 700 may also include a “manual” mode, wherein the controller 118 operates and adjusts blower/fan speed based on user entered settings, e.g., high, medium, low blower/fan speed settings. Other operational data, health data, air quality data, and/or the like may be captured by the device layer 706 and analyzed and/or communicated by the logical layer 704 and/or the application layer 702 to the monitor 102.

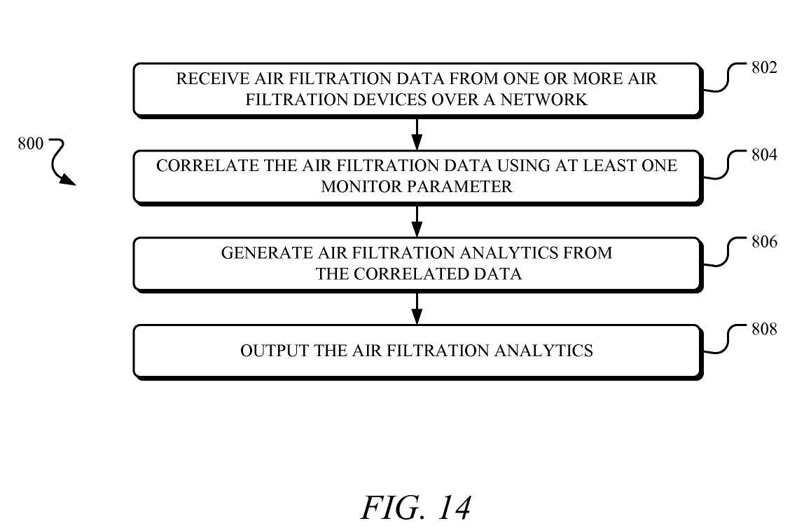

FIG. 14 illustrates example operations 800 for air filtration monitoring. In one implementation, an operation 802 receives air filtration data from one or more air filtration devices over a network. An operation 804 correlates the air filtration data using at least one monitor parameter. An operation 806 generates air filtration analytics from the correlated data, and an operation 808 outputs the air filtration analytics.

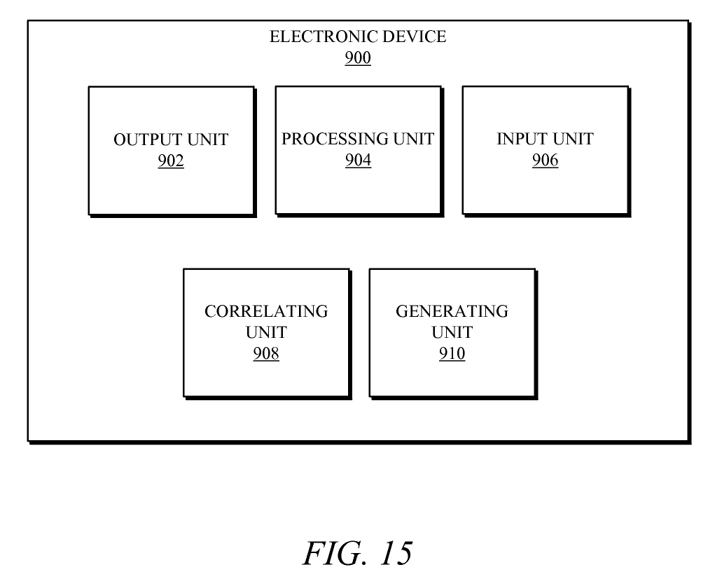

Turning to FIG. 15, an electronic device 900 including operational units 902-910 arranged to perform various operations of the presently disclosed technology is shown. The operational units 902-910 of the device 900 are implemented by hardware or a combination of hardware and software to carry out the principles of the present disclosure. It will be understood by persons of skill in the art that the operational units 902-910 described in FIG. 15 may be combined or separated into sub-blocks to implement the principles of the present disclosure. Therefore, the description herein supports any possible combination or separation or further definition of the operational units 902-910.

In one implementation, the electronic device 900 includes a display unit 902 to display information, such as a graphical user interface, and a processing unit 904 in communication with the display unit 902 and an input unit 906 to receive data from one or more input devices or systems, such as the monitor 102, the air filtration systems 104, and/or the like. Various operations described herein may be implemented by the processing unit 904 using data received by the input unit 906 to output information for display using the display unit 902.

Additionally, in one implementation, the electronic device 900 includes a correlating unit 908 and a generating unit 910. The correlating unit 908 correlates air filtration data captured by one or more of the air filtration systems 104 using at least one monitor parameter. The generating unit 910 generates air filtration analytics from the correlated data.

In another implementation, the electronic device 900 includes units implementing the operations described with respect to FIG. 14. For example, the operation 802 may be implemented by the input unit 906, the operation 804 may be implemented by the correlating unit 908, the operation 806 may be implemented by the generating unit 910, and the operation 808 may be implemented by the output unit 902.

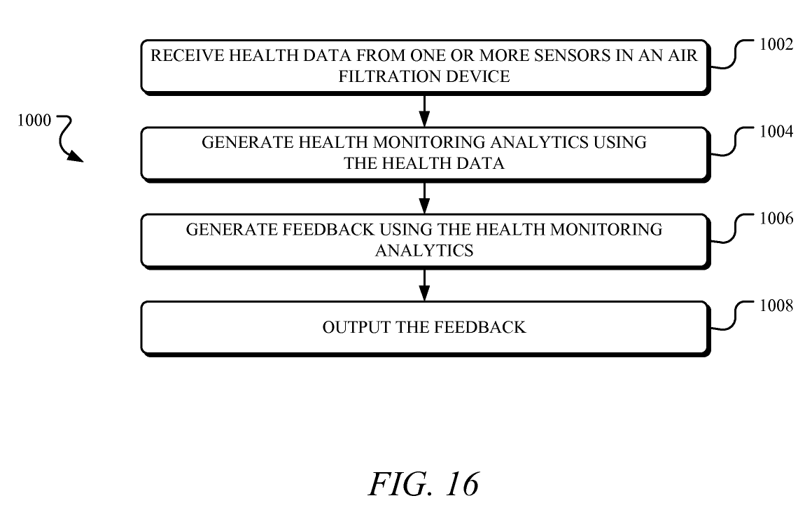

As can be understood from FIG. 16, which illustrates example operations 1000 for health monitoring, in one implementation, an operation 1002 receives health data from one or more sensors in an air filtration device. An operation 1004 generates health monitoring analytics using the health data. An operation 1006 generates feedback using the health monitoring analytics, and an operation 1008 outputs the feedback.

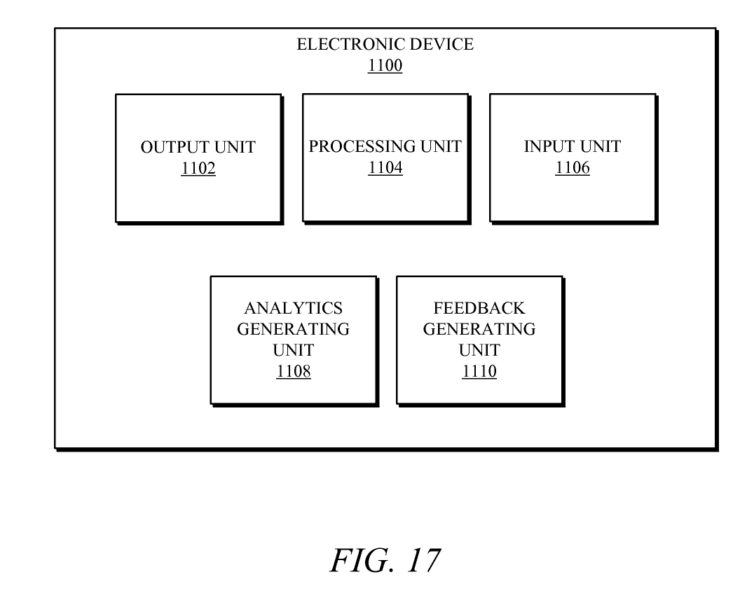

Turning to FIG. 17, an electronic device 1100 including operational units 1102-1110 arranged to perform various operations of the presently disclosed technology is shown. The operational units 1102-1110 of the device 1100 are implemented by hardware or a combination of hardware and software to carry out the principles of the present disclosure. It will be understood by persons of skill in the art that the operational units 1102-1110 described in FIG. 17 may be combined or separated into sub-blocks to implement the principles of the present disclosure. Therefore, the description herein supports any possible combination or separation or further definition of the operational units 1102-1110.

In one implementation, the electronic device 1100 includes a display unit 1102 to display information, such as a graphical user interface, and a processing unit 1104 in communication with the display unit 1102 and an input unit 1106 to receive data from one or more input devices or systems, such as the monitor 102, the air filtration systems 104, and/or the like. Various operations described herein may be implemented by the processing unit 1104 using data received by the input unit 1106 to output information for display using the display unit 1102.

Additionally, in one implementation, the electronic device 1100 includes an analytics generating unit 1108 and a feedback generating unit 1110. The analytics generating unit 1108 generates health monitoring analytics using the health data captured by one or more of the air filtration systems 104. The feedback generating unit 1110 generates feedback using the health monitoring analytics.

In another implementation, the electronic device 1100 includes units implementing the operations described with respect to FIG. 16. For example, the operation 1002 may be implemented by the input unit 1106, the operation 1004 may be implemented by the analytics generating unit 1108, the operation 1006 may be implemented by the feedback generating unit 1110, and the operation 1008 may be implemented by the output unit 1102.

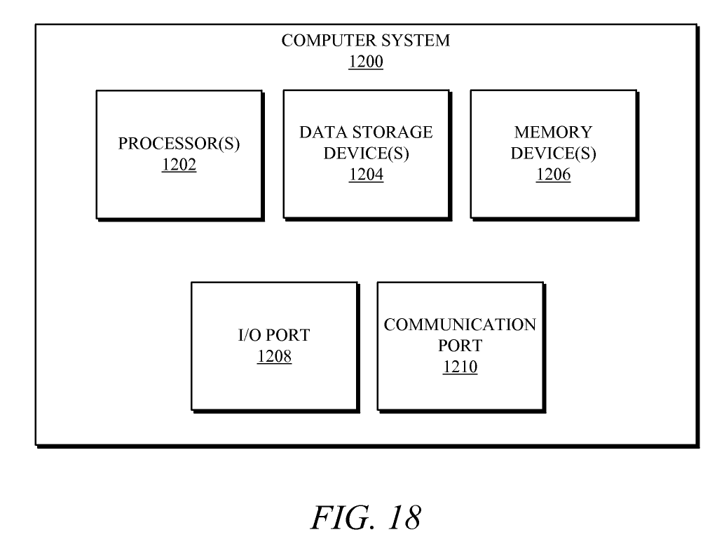

Referring to FIG. 18, a detailed description of an example computing system 1200 having one or more computing units that may implement various systems and methods discussed herein is provided. The computing system 1200 may be applicable to the monitor 102, the controller 118, the server 114, the consumer device 108, the administrator device 110, the user device 212, and other computing or network devices. It will be appreciated that specific implementations of these devices may be of differing possible specific computing architectures not all of which are specifically discussed herein but will be understood by those of ordinary skill in the art.

The computer system 1200 may be a computing system is capable of executing a computer program product to execute a computer process. Data and program files may be input to the computer system 1200, which reads the files and executes the programs therein. Some of the elements of the computer system 1200 are shown in FIG. 18, including one or more hardware processors 1202, one or more data storage devices 1204, one or more memory devices 1208, and/or one or more ports 1208-1210. Additionally, other elements that will be recognized by those skilled in the art may be included in the computing system 1200 but are not explicitly depicted in FIG. 18 or discussed further herein. Various elements of the computer system 1200 may communicate with one another by way of one or more communication buses, point-to-point communication paths, or other communication means not explicitly depicted in FIG. 18.

The processor 1202 may include, for example, a central processing unit (CPU), a microprocessor, a microcontroller, a digital signal processor (DSP), and/or one or more internal levels of cache. There may be one or more processors 1202, such that the processor 1202 comprises a single central-processing unit, or a plurality of processing units capable of executing instructions and performing operations in parallel with each other, commonly referred to as a parallel processing environment.

The computer system 1200 may be a conventional computer, a distributed computer, or any other type of computer, such as one or more external computers made available via a cloud computing architecture. The presently described technology is optionally implemented in software stored on the data stored device(s) 1204, stored on the memory device(s) 1206, and/or communicated via one or more of the ports 1208-1210, thereby transforming the computer system 1200 in FIG. 18 to a special purpose machine for implementing the operations described herein. Examples of the computer system 1200 include personal computers, terminals, workstations, mobile phones, tablets, laptops, personal computers, multimedia consoles, gaming consoles, set top boxes, and the like.

The one or more data storage devices 1204 may include any non-volatile data storage device capable of storing data generated or employed within the computing system 1200, such as computer executable instructions for performing a computer process, which may include instructions of both application programs and an operating system (OS) that manages the various components of the computing system 1200. The data storage devices 1204 may include, without limitation, magnetic disk drives, optical disk drives, solid state drives (SSDs), flash drives, and the like. The data storage devices 1204 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 1206 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 1204 and/or the memory devices 1206, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computer system 1200 includes one or more ports, such as an input/output (I/O) port 1208 and a communication port 1210, for communicating with other computing, network, or vehicle devices. It will be appreciated that the ports 1208-1210 may be combined or separate and that more or fewer ports may be included in the computer system 1200.

The I/O port 1208 may be connected to an I/O device, or other device, by which information is input to or output from the computing system 1200. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing system 1200 via the I/O port 1208. Similarly, the output devices may convert electrical signals received from computing system 1200 via the I/O port 1208 into signals that may be sensed as output by a human, such as sound, light, and/or touch. The input device may be an alphanumeric input device, including alphanumeric and other keys for communicating information and/or command selections to the processor 1202 via the I/O port 1208. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing system 1200 via the I/O port 1208. For example, an electrical signal generated within the computing system 1200 may be converted to another type of signal, and/or vice-versa. In one implementation, the environment transducer devices sense characteristics or aspects of an environment local to or remote from the computing device 1200, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computing device 1200, such as, physical movement of some object (e.g., a mechanical actuator), heating or cooling of a substance, adding a chemical substance, and/or the like.

In one implementation, a communication port 1210 is connected to a network by way of which the computer system 1200 may receive network data useful in executing the methods and systems set out herein as well as transmitting information and network configuration changes determined thereby. Stated differently, the communication port 1210 connects the computer system 1200 to one or more communication interface devices configured to transmit and/or receive information between the computing system 1200 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 1210 to communicate one or more other machines, either directly over a point-to-point communication path, over a wide area network (WAN) (e.g., the Internet), over a local area network (LAN), over a cellular (e.g., third generation (3G) or fourth generation (4G)) network, or over another communication means. Further, the communication port 1210 may communicate with an antenna or other link for electromagnetic signal transmission and/or reception.

In an example implementation, health data, air filtration data, and software and other modules and services may be embodied by instructions stored on the data storage devices 1204 and/or the memory devices 1206 and executed by the processor 1202. The computer system 1200 may be integrated with or otherwise form part of the air filtration system 104.

The system set forth in FIG. 18 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions.

While the present disclosure has been described with reference to various implementations, it will be understood that these implementations are illustrative and that the scope of the present disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A method for monitoring air filtration, the method comprising:
receiving air filtration data from one or more air filtration systems over a network, each of the one or more air filtration systems configured to provide purified air into an enclosed space by removing ultra-fine particles from air using at least one primary filter, the air filtration data captured by one or more sensors;
correlating the air filtration data based on at least one monitoring parameter using at least one computing unit; and
generating air filtration analytics from the correlated data using the at least one computing unit.

2. The method of claim 1, further comprising:
outputting the air filtration analytics for display on a user device.

3. The method of claim 1, wherein the user device is at least one of a consumer device or an administrator device.

4. The method of claim 1, wherein the at least one monitoring parameter includes at least one of: a set of consumers, a captured data type, a behavior pattern, a monitoring area, or an environmental monitoring area.

5. The method of claim 1, wherein the air filtration analytics includes at least one of: respirator analytics, use analytics, health analytics, device analytics, demographic analytics, or media analytics.

6. A system for monitoring air filtration, the system comprising:
one or more air filtration systems configured to capture air filtration data using one or more sensors, each of the one or more air filtration systems configured to provide purified air into an enclosed space by removing ultra-fine particles from air using at least one primary filter; and
at least one computing unit in communication with the one or more air filtration systems over a network, the at least one computing unit generating air filtration analytics from the air filtration data correlated based on at least one monitoring parameter.

7. The system of claim 16, further comprising:
a user device in communication with the at least one computing unit over the network, the user device receiving the air filtration analytics from the at least one computing unit.

8. The system of claim 1, wherein the at least one monitoring parameter includes at least one of: a set of consumers, a captured data type, a behavior pattern, a monitoring area, or an environmental monitoring area.

9. The system of claim 1, wherein the air filtration analytics includes at least one of: respirator analytics, use analytics, health analytics, device analytics, demographic analytics, or media analytics.

10. A method for health monitoring, the method comprising:
receiving health data from a controller in an air filtration system configured to provide purified air into an enclosed space by removing ultra-fine particles from air using at least one primary filter, the health data captured using one or more sensors;
generating health monitoring analytics from the health data using at least one computing unit; and
generating feedback from the health monitoring analytics using the at least one computing unit.

11. The method of claim 10, further comprising:
outputting the feedback to the controller.

12. The method of claim 10, further comprising:
outputting the feedback to at least one of a consumer device or an administrator device.

13. The method of claim 10, wherein the feedback includes at least one of: delivering a drug via the air filtration system, increasing pressure in the enclosed space using the controller, sensing an alert to a user device, or changing an operational parameter of the air filtration system.

14. The method of claim 10, wherein the health monitoring analytics include at least one of: an air flow through the air filtration system; medical condition diagnosis, medical condition monitoring, medical condition testing, or symptoms monitoring.

15. A system for health monitoring, the system comprising:
one or more sensors configured to capture health data, the one or more sensors deployed in an air filtration system configured to provide purified air into an enclosed space by removing ultra-fine particles from air using at least one primary filter; and
at least one computing unit in communication with the one or more sensors, the at least one computing unit generating feedback from health monitoring analytics generated from the health data.

16. The system of claim 15, further comprising:
a user device in communication with the at least one computing unit via a connection.

17. The system of claim 16, wherein the user device is at least one of a consumer device or an administrator device.

18. The system of claim 16, wherein the connection is a wired connection or a wireless connection.

19. The system of claim 15, wherein the feedback includes at least one of: delivering a drug via the air filtration system, increasing pressure in the enclosed space using the controller, sensing an alert to a user device, or changing an operational parameter of the air filtration system.

20. The system of claim 15, wherein the health monitoring analytics include at least one of: an air flow through the air filtration system; medical condition diagnosis, medical condition monitoring, medical condition testing, or symptoms monitoring.
